SEQUENCE LISTING (62 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 21 nt, Homo sapiens
agagguaguagguugcauagu

SEQ ID NO: 5 - RNA, 21 nt, Homo sapiens
ugagguaggagguuguauagu

SEQ ID NO: 6 - RNA, 22 nt, Homo sapiens
ugagguaguagauuguauaguu

SEQ ID NO: 7 - RNA, 21 nt, Homo sapiens
ugagguaguaguuuguacagu

SEQ ID NO: 8 - RNA, 21 nt, Homo sapiens
ugagguaguaguuugugcugu

SEQ ID NO: 9 - RNA, 23 nt, Homo sapiens
agcagcauuguacagggcuauga

SEQ ID NO: 10 - RNA, 24 nt, Homo sapiens
aaaagugcuuacagugcagguagc

SEQ ID NO: 11 - RNA, 21 nt, Homo sapiens
uaaagugcugacagugcagau

SEQ ID NO: 12 - RNA, 23 nt, Homo sapiens
agcagcauuguacagggcuauca

SEQ ID NO: 13 - RNA, 23 nt, Homo sapiens
uacccuguagauccgaauuugug

SEQ ID NO: 14 - RNA, 22 nt, Homo sapiens
uacccuguagaaccgaauuugu

SEQ ID NO: 15 - RNA, 23 nt, Homo sapiens
ucccugagacccuuuaaccugug

SEQ ID NO: 16 - RNA, 22 nt, Homo sapiens
ucccugagacccuaacuuguga

SEQ ID NO: 17 - RNA, 21 nt, Homo sapiens
ucguaccgugaguaauaaugc

SEQ ID NO: 18 - RNA, 22 nt, Homo sapiens
ugagaugaagcacuguagcuca

SEQ ID NO: 19 - RNA, 22 nt, Homo sapiens
ugagaacugaauuccauggguu

SEQ ID NO: 20 - RNA, 22 nt, Homo sapiens
ucucccaacccuuguaccagug

SEQ ID NO: 21 - RNA, 22 nt, Homo sapiens
uagcagcacauaaugguuugug

SEQ ID NO: 22 - RNA, 22 nt, Homo sapiens
uagcagcacaucaugguuuaca

SEQ ID NO: 23 - RNA, 22 nt, Homo sapiens
uagcagcacguaaauauuggcg

SEQ ID NO: 24 - RNA, 24 nt, Homo sapiens
caaagugcuuacagugcagguagu

SEQ ID NO: 25 - RNA, 23 nt, Homo sapiens
aacauucaacgcugucggugagu

SEQ ID NO: 26 - RNA, 22 nt, Homo sapiens
aacauucauugcugucgguggg

SEQ ID NO: 27 - RNA, 22 nt, Homo sapiens
aacauucaaccugucggugagu

SEQ ID NO: 28 - RNA, 22 nt, Homo sapiens
caacggaaucccaaaagcagcu

SEQ ID NO: 29 - RNA, 23 nt, Homo sapiens
cccaguguucagacuaccuguuc

SEQ ID NO: 30 - RNA, 22 nt, Homo sapiens
uacaguagucugcacauugguu

SEQ ID NO: 31 - RNA, 23 nt, Homo sapiens
ugugcaaaucuaugcaaaacuga

SEQ ID NO: 32 - RNA, 23 nt, Homo sapiens
ugugcaaauccaugcaaaacuga

SEQ ID NO: 33 - RNA, 23 nt, Homo sapiens
uaaagugcuuauagugcagguag

SEQ ID NO: 34 - RNA, 22 nt, Homo sapiens
uccuucauuccaccggagucug

SEQ ID NO: 35 - RNA, 22 nt, Homo sapiens
uagcuuaucagacugauguuga

SEQ ID NO: 36 - RNA, 23 nt, Homo sapiens
agcuacauugucugcuggguuuc

SEQ ID NO: 37 - RNA, 24 nt, Homo sapiens
agcuacaucuggcuacugggucuc

SEQ ID NO: 38 - RNA, 21 nt, Homo sapiens
ugucaguuugucaaauacccc

SEQ ID NO: 39 - RNA, 21 nt, Homo sapiens
aucacauugccagggauuucc

SEQ ID NO: 40 - RNA, 21 nt, Homo sapiens
aucacauugccagggauuacc

SEQ ID NO: 41 - RNA, 22 nt, Homo sapiens
uggcucaguucagcaggaacag

SEQ ID NO: 42 - RNA, 21 nt, Homo sapiens
uucaaguaauccaggauaggc

SEQ ID NO: 43 - RNA, 22 nt, Homo sapiens
uucaaguaauucaggauagguu

SEQ ID NO: 44 - RNA, 21 nt, Homo sapiens
uagcaccaucugaaaucgguu

SEQ ID NO: 45 - RNA, 23 nt, Homo sapiens
uagcaccauuugaaaucaguguu

SEQ ID NO: 46 - RNA, 20 nt, Homo sapiens
uagcaccauuugaaaucggu

SEQ ID NO: 47 - RNA, 22 nt, Homo sapiens
cuuucagucggauguuugcagc

SEQ ID NO: 48 - RNA, 22 nt, Homo sapiens
uguaaacauccucgacuggaag

SEQ ID NO: 49 - RNA, 22 nt, Homo sapiens
uguaaacauccuacacucagcu

SEQ ID NO: 50 - RNA, 23 nt, Homo sapiens
uguaaacauccuacacucucagc

SEQ ID NO: 51 - RNA, 22 nt, Homo sapiens
uguaaacauccccgacuggaag

SEQ ID NO: 52 - RNA, 22 nt, Homo sapiens
cuuucagucggauguuuacagc

SEQ ID NO: 53 - RNA, 20 nt, Homo sapiens
uguaaacauccuugacugga

SEQ ID NO: 54 - RNA, 24 nt, Homo sapiens
ucucacacagaaaucgcacccguc

SEQ ID NO: 55 - RNA, 21 nt, Homo sapiens
aucgggaaugucguguccgcc

SEQ ID NO: 56 - RNA, 22 nt, Homo sapiens
uggcaguguauuguuagcuggu

SEQ ID NO: 57 - RNA, 21 nt, Homo sapiens
uauugcacuugucccggccug

SEQ ID NO: 58 - RNA, 22 nt, Homo sapiens
aaagugcuguucgugcagguag

SEQ ID NO: 59 - RNA, 22 nt, Homo sapiens
aacccguagauccgaucuugug

SEQ ID NO: 60 - RNA, 22 nt, Homo sapiens
cacccguagaaccgaccuugcg

SEQ ID NO: 61 - RNA, 22 nt, Homo sapiens
uucacagggaggugucauuuau

SEQ ID NO: 62 - RNA, 24 nt, Homo sapiens
ugaaacauacacgggaaaccucuu

(19) United States
(12) Patent Application Publication
(10) Pub. No.: US 2009/0092974 A1
(43) Pub. Date: Apr. 9, 2009

(54) Micrornas differentially expressed in leukemia and uses thereof

(75) Inventors: Tim Davison, Hillsborough (GB); Dmitriy Ovcharenko, Austin, TX (US); Jeffrey Shelton, Buda, TX (US); Kevin Kelnar, Kyle, TX (US); David Brown, Austin, TX (US)

Correspondence Address: Fullbright & Jaworski L.L.P., 600 Congress Avenue, Suite 2400, Austin, TX, 78701, US

(73) Assignee: ASURAGEN, INC., Austin, TX (US)

(21) Appl. No.: 11/953,606

(22) Filed: Dec. 10, 2007

Related U.S. Application Data
(60) Provisional application No. 60/869,295, filed on Dec. 8, 2006.

(51) Int. Cl. C12Q 1/68

(52) U.S. Cl. 435/6

(57) ABSTRACT

The present invention concerns methods and compositions for identifying a miRNA profile for acute myeloid leukemia (AML), and using the profile in assessing the condition of a patient.

This application is related to U.S. Provisional Patent Application 60/869,295 filed on Dec. 8, 2006, which is hereby incorporated by reference in its entirety.

### BACKGROUND OF THE INVENTION

I. Field of the Invention

The present invention relates generally to the field of molecular biology. More particularly, it concerns methods and compositions involving microRNA (miRNAs) molecules. Certain aspects of the invention include applications for miRNAs in diagnostics, therapeutics, and prognostics of acute myeloid leukemia.

II. Background

In 2001, several groups used a cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are distinct.

miRNAs thus far observed have been approximately 21-22 nucleotides in length and they arise from longer precursors, which are transcribed from non-protein-encoding genes. See review of Carrington et al. (2003). The precursors form structures that fold back on themselves in self-complementary regions; they are then processed by the nuclease Dicer in animals or DCL1 in plants. miRNA molecules interrupt translation through precise or imprecise base-pairing with their targets.

Many miRNAs are conserved among diverse organisms, and this has led to the suggestion that miRNAs are involved in essential biological processes throughout the life span of an organism (Esquela-Kerscher and Slack, 2006). In particular, miRNAs have been implicated in regulating cell growth, and cell and tissue differentiation; cellular processes that are associated with the development of cancer. For instance, lin-4 and let-7 both regulate passage from one larval state to another during C. elegans development (Ambros, 2001). mir-14 and bantam are Drosophila miRNAs that regulate cell death, apparently by regulating the expression of genes involved in apoptosis (Brennecke et al., 2003, Xu et al., 2003).

Research on miRNAs is increasing as scientists are beginning to appreciate the broad role that these molecules play in the regulation of eukaryotic gene expression. In particular, several recent studies have shown that expression levels of numerous miRNAs are associated with various cancers (reviewed in Esquela-Kerscher and Slack, 2006). Reduced expression of two miRNAs correlates strongly with chronic lymphocytic leukemia in humans, providing a possible link between miRNAs and cancer (Calin et al., 2002). Others have evaluated the expression patterns of large numbers of miRNAs in multiple human cancers and observed differential expression of almost all miRNAs across numerous cancer types (Lu et al., 2005). Most studies link miRNAs to cancer only by indirect evidence. However, He et al. (2005) has provided more direct evidence that miRNAs may contribute directly to causing cancer by forcing the over-expression of six miRNAs in mice that resulted in a significant increase in B cell lymphomas.

One human cancer that is in need of additional diagnostic and prognostic compositions and methods is acute myeloid leukemia (AML). AML is a genetically and phenotypically heterogeneous disorder of the hematopoietic stem cells that is characterized by failure of blood cells to differentiate and by proliferation of stem cells. Existing cytogenetic methods for identifying chromosomal aberrations are often inadequate for clearly defining the stage of AML in patients or for defining the aggressiveness of the disease. Furthermore, although genetic markers for identifying AML patients with a normal karyotype are currently under investigation (Mrozek et al., 2006), effective diagnostic methods for these patients have remained elusive. A need exists for additional diagnostic and prognostic assays that can identify AML in patients with a normal karyotype and for prognostic assays that can identify the stage or progression of AML in patients and that can be used to formulate an appropriate therapeutic response in various patients.

### SUMMARY OF THE INVENTION

The present invention overcomes these problems in the art by identifying miRNAs that are differentially expressed or mis-regulated in various states of normal, cancerous, and/or abnormal tissues, including but not limited to normal blood cells, and blood cells from patients having, suspected of having, or at risk of developing acute myeloid leukemia. Further, the invention describes a method for diagnosing and/or prognosing acute myeloid leukemia that is based on determining levels (increased or decreased) of selected miRNAs in patient-derived samples.

The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term can be used to refer to the single-stranded RNA molecule processed from a precursor or in certain instances the precursor itself. Individual miRNAs have been identified and sequenced in different organisms, and they have been given names. Names of miRNAs and their sequences related to the present invention are provided herein. The methods and compositions should not be limited to miRNAs identified in the application, as they are provided as examples, not necessarily as limitations of the invention.

In some embodiments, it may be useful to know whether a cell expresses a particular miRNA endogenously or whether such expression is affected under particular conditions or when it is in a particular disease state. Thus, in some embodiments of the invention, methods include assaying a cell or a sample containing a cell for the presence of one or more miRNA. Consequently, in some embodiments, methods include a step of generating a miRNA profile for a sample. The term “miRNA profile” refers to a set of data regarding the expression pattern for one or more miRNAs (e.g., a plurality of miRNA from Table 1) in the sample; it is contemplated that the miRNA profile can be obtained using a set of miRNAs, using for example nucleic acid amplification or hybridization techniques well know to one of ordinary skill in the art. The difference in the expression profile in the sample from the patient and a reference expression profile, such as an expression profile from a normal or non-pathologic sample, is indicative of a pathologic, disease, or cancerous condition. An miRNA or probe set comprising or identifying a segment of a corresponding miRNA can include all or part of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or any integer or range derivable there between, of a miRNA or a probe listed in Table 1.

Embodiments of the invention are directed to compositions and methods for assessing a pathological condition in a patient comprising measuring an expression profile of one or more miRNA in a sample from the patient, wherein a difference in the expression profile in the sample from the patient and an expression profile of a normal sample or reference expression profile is indicative of acute myeloid leukemia (AML). In certain aspects of the invention, the miRNA is one or more of miR-29, miR-126, miR-21, miR-99, miR-125, miR-16, miR-20, miR-10, miR-199a-AS, miR-23, let-7, miR-106, miR-181, miR-15, miR-223, miR-425, miR-449, miR-513, miR-494, miR-17-5p, miR-221, mir-19, miR-92, miR-222, miR-27a, miR-24, miR-26, miR-191, miR-30, miR-103, miR-107, miR-146, miR-30a-5p, miR-93, miR-342, miR-150, miR-205, miR-143, or miR-199a, including any combination thereof.

In some embodiments miRNAs include all miRNA of Table 1, except miR-223, mir-15a, miR-16, miR-203, miR-103, miR-23b, miR-107, miR-17-5p, mir-23a, miR-335, miR-150, miR-20, miR-17, miR-25, miR-29b and/or miR-191. In other embodiments miR-223, mir-15a, miR-16, miR-203, miR-103, miR-23b, miR-107, miR-17-5p, mir-23a, miR-335, miR-150, miR-20, miR-17, miR-25, miR-29b and/or miR-191 are not used in the compositions and/or methods of the invention. In further embodiments it is a miRNA disclosed herein that is indicative of AML, other than miR-223, mir-15a, miR-16, miR-203, miR-103, miR-23b, miR-107, miR-17-5p, mir-23a, miR-335, miR-150, miR-20, miR-17, miR-25, miR-29b and miR-191. In still other embodiments, miR-223, mir-15a, miR-16, miR-203, miR-103, miR-23b, miR-107, miR-17-5p, mir-23a, miR-335, miR-150, miR-20, miR-17, miR-25, miR-29b and/or miR191 are used in combination with one or more other miRNA selected from Table 1.

In certain aspects, the sample is a blood or tissue sample. Typically, the sample will be enriched for or comprise CD34+ cells.

In further aspects, the difference in expression profile is indicative of a cytogenetic sub-classification of AML including, but not limited to monosomy 7/deletion 7, inv (16), or normal cytogenetic sub-classification.

In certain embodiments, the reduced expression of one or more miRNA is indicative of an inv (16) cytogenetic sub-classification, wherein the miRNA is one or more of hsa-miR-29, miR-126, miR-21, miR-99, miR-125, miR-16, miR-20, miR-10, miR-199a-AS, miR-23, let-7, miR-106, miR-181, miR-15, or miR-26. The increased expression of one or more miRNA can be indicative of an inv (16) cytogenetic sub-classification, wherein the miRNA is miR-223, miR-425, or both miR-223 and miR-425.

In a further embodiment, the reduced expression of one or more miRNA is indicative of a monosomy 7/deletion 7 cytogenetic sub-classification, wherein the miRNA is one or more of miR-449, miR-513, or miR-494. The increased expression of one or more miRNA also can be indicative of a monosomy 7/deletion 7 cytogenetic sub-classification, wherein the miRNA is one or more of miR-181, miR-126, miR-17-5p, miR-20, miR-23, miR-221, miR-106, mir-19, miR-21, miR-92, miR-16, miR-222, miR-223, miR-27a, miR-24, miR-26, miR-191, miR-30, miR-103, let-7, miR-15, miR-107, miR-146, miR-30a-5p, miR-93, or miR-29.

In still a further embodiment, the reduced expression of one or more miRNA is indicative of a normal cytogenetic sub-classification, wherein the miRNA is one or more of miR-223, miR-16, miR-23, miR-342, miR-17-5p, miR-15, miR-150, miR-103, miR-107, miR-106, or miR-20. The increased expression of one or more miRNA also can be indicative of a normal cytogenetic sub-classification, wherein the miRNA is one or more of miR-205, miR-181, miR-125, miR-143, or miR-199a.

Aspects of the invention can be used to diagnose or assess a patient's condition. For example, the methods can be used to screen for a pathological condition, assess prognosis of a pathological condition, stage a pathological condition, or assess response of a pathological condition to therapy. In particular aspects, assessing the pathological condition of the patient can be assessing prognosis of the AML patient. Prognosis may include, but is not limited to an estimation of the time or expected time of survival, assessment of response to a therapy, and the like. In certain aspects, the altered expression of one or more miRNA is prognostic for a patient having AML, wherein the miRNA is one or more of miR-199a-AS, miR-23, miR-26, miR-148, miR-28, let 71, miR-191, miR-146, miR-422, miR-297, miR-128, miR-93, miR-105, miR-25, miR-150, miR-184, miR-100, miR-199a, miR-148, miR-105, miR-195, miR-341, miR-30a-AS, miR-145, miR-205, miR-126, miR-221, miR-342, miR-335, miR-106, miR-16, miR-22, miR-139, miR-223, or miR-10a.

In further aspects, the altered expression of one or more miRNA is prognostic for a patient having monosomy 7/deletion 7 cytogenetic sub-classification, wherein the miRNA is one or more of miR-199a-AS, miR-26, miR-148, miR-28, let 71, miR-191, miR-146, miR-422, miR-297, miR-128, miR-93, miR-105, or miR-25. In still further aspects, the altered expression of one or more miRNA is prognostic for a patient having normal cytogenetic sub-classification, wherein the miRNA is one or more of miR-23. miR-150, miR-184, miR-100, miR-199a, miR-148, miR-105, miR-195, miR-341, miR-30a-AS, miR-199a-AS, miR-145, miR-205, miR-126, miR-221, miR-342, miR-335, miR-106, miR-16, miR-22, miR-139, miR-26, miR-223, or miR-10a.

Certain embodiments of the invention include determining expression of one or more miRNA by using an amplification assay or a hybridization assay, a variety of which are well known to one of ordinary skill in the art. In certain aspects, an amplification assay can be a quantitative amplification assay, such as quantitative RT-PCR or the like. In still further aspects, a hybridization assay can include array hybridization assays or solution hybridization assays. The miRNA may be labeled from the sample and/or hybridizing the labeled miRNA to one or more miRNA probes. Nucleic acids, miRNA, and/or miRNA probes may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art.

The present invention also concerns kits containing compositions of the invention or compositions to implement methods of the invention. In some embodiments, kits can be used to evaluate one or more miRNA molecules. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or more miRNA probes, synthetic miRNA molecules or miRNA inhibitors, or any range and combination derivable therein. In some embodiments, there are kits for evaluating miRNA activity in a cell. Kits may comprise components, which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more. Kits for using miRNA probes, synthetic miRNAs, nonsynthetic, and/or miRNA inhibitors of the invention for therapeutic, prognostic, or diagnostic applications are included as part of the invention. Specifically contemplated are any such molecules corresponding to any miRNA reported to influence biological activity, such as those discussed herein. In certain aspects, negative and/or positive control synthetic miRNAs and/or miRNA inhibitors are included in some kit embodiments. The control molecules can be used to verify transfection efficiency and/or control for transfection-induced changes in cells. Certain embodiments are directed to a kit for assessment of an AML patient by miRNA profiling of a sample comprising, in suitable container means, two or more miRNA hybridization or amplification reagents comprising one or more of miR-29, miR-126, miR-21, miR-99, miR-125, miR-16, miR-20, miR-10, miR-199a-AS, miR-23, let-7, miR-106, miR-181, miR-15, miR-223, miR-425, miR-449, miR-513, miR-494, miR-17-5p, miR-221, miR-19, miR-92, miR-222, miR-27a, miR-24, miR-26, miR-191, miR-30, miR-103, miR-107, miR-146, miR-30a-5p, miR-93, miR-342, miR-150, miR-205, miR-143, or miR-199a. The kit can comprise reagents for labeling miRNA in a sample and/or miRNA hybridization reagents. The miRNA hybridization reagents typically comprise hybridization probes. miRNA amplification reagents include, but are not limited to amplification primers.

In some embodiments of the invention, a miRNA profile is generated by steps that include: (a) labeling miRNA in the sample; (b) hybridizing miRNA to a number of probes, or amplifying a number of miRNA, and (c) determining miRNA hybridization to the probes or detecting miRNA amplification products, wherein a miRNA profile is generated. See U.S. Provisional Patent Application 60/575,743 and the U.S. Provisional Patent Application 60/649,584, and U.S. patent application Ser. No. 11/141,707, all of which are hereby incorporated by reference.

Methods of the invention involve diagnosing and/or assessing the prognosis of a patient based on a miRNA expression profile. In certain embodiments, the elevation or reduction in the level of expression of a particular miRNA or set of miRNA in a cell is correlated with a disease state compared to the expression level of that miRNA or set of miRNA in a normal cell. This correlation allows for diagnostic and/or prognostic methods to be carried out when the expression level of a miRNA is measured in a biological sample being assessed and then compared to the expression level of a normal cell. It is specifically contemplated that miRNA profiles for patients, particularly those suspected of having a particular disease or condition such as AML, can be generated by evaluating any of or sets of the miRNAs discussed in this application. The miRNA profile that is generated from the patient will be one that provides information regarding the particular disease or condition. In many embodiments, the miRNA profile is generated using miRNA hybridization or amplification, (e.g., array hybridization or RT-PCR). In certain aspects, a miRNA profile can be used in conjunction with other diagnostic and/or prognostic tests, such protein profiles in the serum, e.g., CA19-9 detection, or cytogenetic assessment.

The methods can further comprise one or more of the steps including: (a) obtaining a sample from the patient, (b) isolating nucleic acids from the sample, (c) labeling the nucleic acids isolated from the sample, and (d) hybridizing the labeled nucleic acids to one or more probes. Nucleic acids of the invention include one or more nucleic acid comprising at least one segment having a sequence or complementary sequence of one or more of the miRNA sequences in Table 1. In certain aspects, the nucleic acids identify one or more miRNAs listed in Table 1.

It is contemplated that any method or composition described herein can be implemented with respect to any other method or composition described herein and that different embodiments may be combined. It is specifically contemplated that any methods and compositions discussed herein with respect to miRNA molecules or miRNA may be implemented with respect to synthetic miRNAs. In some embodiments the synthetic miRNA is exposed to the proper conditions to allow it to become a mature miRNA under physiological circumstances. The claims originally filed are contemplated to cover claims that are multiply dependent on any filed claim or combination of filed claims.

Also, any embodiment of the invention involving specific miRNAs by name is contemplated also to cover embodiments involving miRNAs whose sequences are at least 80, 81, 82, 83, 84, 85, 86, 87, 88, 89, 90, 91, 92, 93, 94, 95, 96, 97, 98, 99% identical to the mature sequence of the specified miRNA.

TABLE 1

Listing of miRNA probes for sample assessment.

| | | | miR Base |
|---|---|---|---|
| Probe Sequence | SEQ ID NO: | miRNA | Information |
| UGAGGUAGUAGGUUGUAUAGUU | SEQ ID NO: 1 | hsa-let-7a | >hsa-let-7a |
| | | | MIMAT0000062 |
| UGAGGUAGUAGGUUGUGUGGUU | SEQ ID NO: 2 | hsa-let-7b | >hsa-let-7b |
| | | | MIMAT0000063 |
| UGAGGUAGUAGGUUGUAUGGUU | SEQ ID NO: 3 | hsa-let-7c | >hsa-let-7c |
| | | | MIMAT0000064 |
| AGAGGUAGUAGGUUGCAUAGU | SEQ ID NO: 4 | hsa-let-7d | >hsa-let-7d |
| | | | MIMAT0000065 |
| UGAGGUAGGAGGUUGUAUAGU | SEQ ID NO: 5 | hsa-let-7e | >hsa-let-7e |
| | | | MIMAT0000066 |
| UGAGGUAGUAGAUUGUAUAGUU | SEQ ID NO: 6 | hsa-let-7f | >hsa-let-7f |
| | | | MIMAT0000067 |
| UGAGGUAGUAGUUUGUACAGU | SEQ ID NO: 7 | hsa-let-7g | >hsa-let-7g |
| | | | MIMAT0000414 |
| UGAGGUAGUAGUUUGUGCUGU | SEQ ID NO: 8 | hsa-let-7i | >hsa-let-7i |
| | | | MIMAT0000415 |
| AGCAGCAUUGUACAGGGCUAUGA | SEQ ID NO: 9 | hsa-miR-103 | >hsa-miR-103 |
| | | | MIMAT0000101 |
| AAAAGUGCUUACAGUGCAGGUAGC | SEQ ID NO: 10 | hsa-miR-106a | >hsa-miR-106a |
| | | | MIMAT0000103 |
| UAAAGUGCUGACAGUGCAGAU | SEQ ID NO: 11 | hsa-miR-106b | >hsa-miR-106b |
| | | | MIMAT0000680 |
| AGCAGCAUUGUACAGGGCUAUCA | SEQ ID NO: 12 | hsa-miR-107 | >hsa-miR-107 |
| | | | MIMAT0000104 |
| UACCCUGUAGAUCCGAAUUUGUG | SEQ ID NO: 13 | hsa-miR-10a | >hsa-miR-10a |
| | | | MIMAT0000253 |
| UACCCUGUAGAACCGAAUUUGU | SEQ ID NO: 14 | hsa-miR-10b | >hsa-miR-10b |
| | | | MIMAT0000254 |
| UCCCUGAGACCCUUUAACCUGUG | SEQ ID NO: 15 | hsa-miR-125a | >hsa-miR-125a |
| | | | MIMAT0000443 |
| UCCCUGAGACCCUAACUUGUGA | SEQ ID NO: 16 | hsa-miR-125b | >hsa-miR-125b |
| | | | MIMAT0000423 |
| UCGUACCGUGAGUAAUAAUGC | SEQ ID NO: 17 | hsa-miR-126 | >hsa-miR-126 |
| | | | MIMAT0000445 |
| UGAGAUGAAGCACUGUAGCUCA | SEQ ID NO: 18 | hsa-miR-143 | >hsa-miR-143 |
| | | | MIMAT0000435 |
| UGAGAACUGAAUUCCAUGGGUU | SEQ ID NO: 19 | hsa-miR-146a | >hsa-miR-146a |
| | | | MIMAT0000449 |
| UCUCCCAACCCUUGUACCAGUG | SEQ ID NO: 20 | hsa-miR-150 | >hsa-miR-150 |
| | | | MIMAT0000451 |
| UAGCAGCACAUAAUGGUUUGUG | SEQ ID NO: 21 | hsa-miR-15a | >hsa-miR-15a |
| | | | MIMAT0000068 |
| UAGCAGCACAUCAUGGUUUACA | SEQ ID NO: 22 | hsa-miR-15b | >hsa-miR-15b |
| | | | MIMAT0000417 |
| UAGCAGCACGUAAAUAUUGGCG | SEQ ID NO: 23 | hsa-miR-16 | >hsa-miR-16 |
| | | | MIMAT0000069 |
| CAAAGUGCUUACAGUGCAGGUAGU | SEQ ID NO: 24 | hsa-miR-17-5p | >hsa-miR-17-5p |
| | | | MIMAT0000070 |
| AACAUUCAACGCUGUCGGUGAGU | SEQ ID NO: 25 | hsa-miR-181a | >hsa-miR-181a |
| | | | MIMAT0000256 |
| AACAUUCAUUGCUGUCGGUGGG | SEQ ID NO: 26 | hsa-miR-181b | >hsa-miR-181b |
| | | | MIMAT0000257 |
| AACAUUCAACCUGUCGGUGAGU | SEQ ID NO: 27 | hsa-miR-181c | >hsa-miR-181c |
| | | | MIMAT0000258 |
| CAACGGAAUCCCAAAAGCAGCU | SEQ ID NO: 28 | hsa-miR-191 | >hsa-miR-191 |
| | | | MIMAT0000440 |
| CCCAGUGUUCAGACUACCUGUUC | SEQ ID NO: 29 | hsa-miR-199a | >hsa-miR-199a |
| | | | MIMAT0000231 |
| UACAGUAGUCUGCACAUUGGUU | SEQ ID NO: 30 | hsa-miR-199a-AS | >hsa-miR-199a* |
| | | | MIMAT0000232 |
| UGUGCAAAUCUAUGCAAAACUGA | SEQ ID NO: 31 | hsa-miR-19a | >hsa-miR-19a |
| | | | MIMAT0000073 |
| UGUGCAAAUCCAUGCAAAACUGA | SEQ ID NO: 32 | hsa-miR-19b | >hsa-miR-19b |
| | | | MIMAT0000074 |
| UAAAGUGCUUAUAGUGCAGGUAG | SEQ ID NO: 33 | hsa-miR-20a | >hsa-miR-20a |
| | | | MIMAT0000075 |
| UCCUUCAUUCCACCGGAGUCUG | SEQ ID NO: 34 | hsa-miR-205 | >hsa-miR-205 |
| | | | MIMAT0000266 |
| UAGCUUAUCAGACUGAUGUUGA | SEQ ID NO: 35 | hsa-miR-21 | >hsa-miR-21 |
| | | | MIMAT0000076 |
| AGCUACAUUGUCUGCUGGGUUUC | SEQ ID NO: 36 | hsa-miR-221 | >hsa-miR-221 |
| | | | MIMAT0000278 |
| AGCUACAUCUGGCUACUGGGUCUC | SEQ ID NO: 37 | hsa-miR-222 | >hsa-miR-222 |
| | | | MIMAT0000279 |
| UGUCAGUUUGUCAAAUACCCC | SEQ ID NO: 38 | hsa-miR-223 | >hsa-miR-223 |
| | | | MIMAT0000280 |
| AUCACAUUGCCAGGGAUUUCC | SEQ ID NO: 39 | hsa-miR-23a | >hsa-miR-23a |
| | | | MIMAT0000078 |
| AUCACAUUGCCAGGGAUUACC | SEQ ID NO: 40 | hsa-miR-23b | >hsa-miR-23b |
| | | | MIMAT0000418 |
| UGGCUCAGUUCAGCAGGAACAG | SEQ ID NO: 41 | hsa-miR-24 | >hsa-miR-24 |
| | | | MIMAT0000080 |
| UUCAAGUAAUCCAGGAUAGGC | SEQ ID NO: 42 | hsa-miR-26a | >hsa-miR-26a |
| | | | MIMAT0000082 |
| UUCAAGUAAUUCAGGAUAGGUU | SEQ ID NO: 43 | hsa-miR-26b | >hsa-miR-26b |
| | | | MIMAT0000083 |
| UAGCACCAUCUGAAAUCGGUU | SEQ ID NO: 44 | hsa-miR-29a | >hsa-miR-29a |
| | | | MIMAT0000086 |
| UAGCACCAUUUGAAAUCAGUGUU | SEQ ID NO: 45 | hsa-miR-29b | >hsa-miR-29b |
| | | | MIMAT0000100 |
| UAGCACCAUUUGAAAUCGGU | SEQ ID NO: 46 | hsa-miR-29c | >hsa-miR-29c |
| | | | MIMAT0000681 |
| CUUUCAGUCGGAUGUUUGCAGC | SEQ ID NO: 47 | hsa-miR-30a-3p | >hsa-miR-30a-3p |
| | | | MIMAT0000088 |
| UGUAAACAUCCUCGACUGGAAG | SEQ ID NO: 48 | hsa-miR-30a-5p | >hsa-miR-30a-5p |
| | | | MIMAT0000087 |
| UGUAAACAUCCUACACUCAGCU | SEQ ID NO: 49 | hsa-miR-30b | >hsa-miR-30b |
| | | | MIMAT0000420 |
| UGUAAACAUCCUACACUCUCAGC | SEQ ID NO: 50 | hsa-miR-30c | >hsa-miR-30c |
| | | | MIMAT0000244 |
| UGUAAACAUCCCCGACUGGAAG | SEQ ID NO: 51 | hsa-miR-30d | >hsa-miR-30d |
| | | | MIMAT0000245 |
| CUUUCAGUCGGAUGUUUACAGC | SEQ ID NO: 52 | hsa-miR-30e-3p | >hsa-miR-30e-3p |
| | | | MIMAT0000693 |
| UGUAAACAUCCUUGACUGGA | SEQ ID NO: 53 | hsa-miR-30e-5p | >hsa-miR-30e-5p |
| | | | MIMAT0000692 |
| UCUCACACAGAAAUCGCACCCGUC | SEQ ID NO: 54 | hsa-miR-342 | >hsa-miR-342 |
| | | | MIMAT0000753 |
| AUCGGGAAUGUCGUGUCCGCC | SEQ ID NO: 55 | hsa-miR-425 | >hsa-miR-425-3p |
| | | | MIMAT0001343 |
| UGGCAGUGUAUUGUUAGCUGGU | SEQ ID NO: 56 | hsa-miR-449 | >hsa-miR-449 |
| | | | MIMAT0001541 |
| UAUUGCACUUGUCCCGGCCUG | SEQ ID NO: 57 | hsa-miR-92 | >hsa-miR-92 |
| | | | MIMAT0000092 |
| AAAGUGCUGUUCGUGCAGGUAG | SEQ ID NO: 58 | hsa-miR-93 | >hsa-miR-93 |
| | | | MIMAT0000093 |
| AACCCGUAGAUCCGAUCUUGUG | SEQ ID NO: 59 | hsa-miR-99a | >hsa-miR-99a |
| | | | MIMAT0000097 |
| CACCCGUAGAACCGACCUUGCG | SEQ ID NO: 60 | hsa-miR-99b | >hsa-miR-99b |
| | | | MIMAT0000689 |
| UUCACAGGGAGGUGUCAUUUAU | SEQ ID NO: 61 | hsa-miR-513 | >hsa-miR-513 |
| | | | MIMAT0002877 |
| UGAAACAUACACGGGAAACCUCUU | SEQ ID NO: 62 | hsa-miR-494 | >hsa-miR-494 |
| | | | MIMAT0002816 |

It will be further understood that shorthand notations are employed such that a generic description of a miRNA refers to any of its gene family members (distinguished by a number), unless otherwise indicated. It is understood by those of skill in the art that a “gene family” refers to a group of genes having the same miRNA coding sequence. Typically, members of a gene family are identified by a number following the initial designation. For example, miR-16-1 and miR-16-2 are members of the miR-16 gene family and “mir-7” refers to miR-7-1, miR-7-2 and miR-7-3. Moreover, unless otherwise indicated, a shorthand notation refers to related miRNAs (distinguished by a letter). Thus, “let-7,” for example, refers to let-7a, let-7b, let-7c, let-7d, let-7e, and the like. Exceptions to these shorthand notations will be otherwise identified.

Other embodiments of the invention are discussed throughout this application. Any embodiment discussed with respect to one aspect of the invention applies to other aspects of the invention as well and vice versa. The embodiments in the Example and Detailed Description section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C Survival curves (Kaplan-Meier) demonstrating the relationship with expression of an individual miRNA and patient survival. (FIG. 1A) miR-26, generated from array data; (FIG. 1B) miR-26, generated from PCR data; (FIG. 1C) miR-146, generated from array data.

FIGS. 2A-2C Survival curves (Kaplan-Meier), generated from array data, demonstrating the relationship with expression of an individual miRNA and patient survival. (FIG. 2A) miR-184; (FIG. 2B) miR-105; (FIG. 2C) miR-10a.

FIGS. 3A-3B. Survival curves (Kaplan-Meier), generated from array data, demonstrating the relationship with expression of an individual miRNA and patient survival. (FIG. 3A) miR-148 (FIG. 3B) miR-150.

### DETAILED DESCRIPTION OF THE INVENTION

The present invention is directed to compositions and methods relating to preparation and characterization of miRNAs, as well as use of miRNAs for therapeutic, prognostic, and diagnostic applications, particularly those methods and compositions related to assessing and/or identifying acute myeloid leukemia (AML).

Acute myeloid leukemia is a genetically and phenotypically heterogeneous disorder of the hematopoietic stem cells that is characterized by failure of blood cells to differentiate and by proliferation of stem cells. These anomalies result in accumulation of non-functional blood cells called myeloblasts. Over 23,000 cases of AML are diagnosed each year in the United States, making it the most common form of leukemia in adults. More than 70% of AML patients will succumb to the disease every year, and only 15% of adults with AML are expected to survive at least 3 years (Hiddemann et al. 1999; Goldstone et al. 2001).

AML results from chromosomal aberrations in the stem cells that give rise to the leukemia cells, and the nature of these chromosomal changes defines how well a patient will respond to therapy. Aberrations of chromosome 16 (inversion 16 or inv (16)) and of chromosome 7 (loss of the long arm, 7q (7q−) or loss of one entire chromosome 7 (monosomy 7 or −7) are the two most common causes of AML that can be detected by cytogenetics. Patients with the inv (16) aberration usually achieve remission after induction chemotherapy and have a low probability of relapse (Marcucci et al., 2005). In contrast, AML patients with partial or complete loss of chromosome 7 (monosomy 7 or 7q−) rarely achieve remission or relapse rapidly (Byrd et al., 2002; Grimwade, 2001). Inv (16) or monosomy 7/7q− are observed in approximately 50% of AML patients. The remaining AML patients fall into an intermediate risk group of patients that are characterized by a relatively normal karyotype.

The miRNA expression profiles of the CD34+ cell populations were assessed for more than fifty AML patients with three different karyotypes (monosomy 7/deletion 7, inv (16), and normal karyotype) and variable survival profiles. The expression levels for the various miRNAs were compared between the leukemia patients and individuals with no signs of the disease. These data were used to identify miRNAs whose altered expression correlates with each of the three different karyotypes. Any one of these miRNAs or a combination of these miRNAs could be used as diagnostic analytes to determine if an individual has AML and to further define the chromosomal abnormality that resulted in the disease.

Reduced expression (relative to normal) of the any one or a combination of hsa-miR-29, miR-126, miR-21, miR-99, miR-125, miR-16, miR-20, miR-10, miR-199a-AS, miR-23, let-7, miR-106, miR-181, miR-15, and/or miR-26, and/or increased expression (relative to normal) of any one or a combination of hsa-miR-223 or hsa-miR-425 in a blood sample enriched in CD34+ cells is indicative of an individual with AML harboring the inv(16) translocation. Reduced expression (relative to normal) of the any one or a combination of miR-449, miR-513, and/or miR-494, and/or increased expression (relative to normal) of any one or a combination of miR-181, miR-126, miR-17-5p, miR-20, miR-23, miR-221, miR-106, miR-19, miR-21, miR-92, miR-16, miR-222, miR-223, miR-27a, miR-24, miR-26, miR-191, miR-30, miR-103, let-7, miR-15, miR-107, miR-146, miR-30a-5p, miR-93, miR-29, and/or miR-106 in a blood sample containing CD34+ cells is indicative of an individual with AML harboring an abnormality of chromosome 7. Reduced expression (relative to normal) of the any one or a combination of miR-223, miR-16, miR-23, miR-342, miR-17-5p, miR-15, miR-150, miR-103, miR-107, miR-106, and/or miR-20, and/or increased expression (relative to normal) of any one or a combination of miR-205, miR-181, miR-125, miR-143, and/or miR-199a in a blood sample enriched in CD34+ cells is indicative of an individual with AML who harbors a relatively normal karyotype.

Diagnostic and/or prognostic assays featuring any one or combination of the miRNAs listed above could be used to diagnose an AML patient with any one or combination of translocation types. The absolute values that define the normal and leukemia patient populations will depend upon the platform that is used for quantifying the miRNA levels. Potential platforms include, but are not restricted to, quantitative, competitive, or relative RT-PCR; quantitative or semi-quantitative array analysis; a bead-based hybridization assay such as ones that feature the Luminex platform; signal amplification methods like the Invader assay (Third Wave Technologies); and direct hybridization approaches like in situ hybridization, Northern analysis, HPA (GenProbe), B-DNA (Digene), ribonuclease protection assay, and others.

For each group of patients exhibiting a given karyotype, the data may be further assessed to identify those miRNAs whose expression correlated with a poor prognosis or propensity to respond to a particular therapeutic regimen. For all three patient types, there were miRNAs whose expression levels correlated with prognosis. These particular miRNAs could be used independently or in combination as analytes in diagnostic assays to determine the prognosis for a patient. A physician could use this information to prescribe treatment that would be best suited for the patient.

Altered expression of miR-199a-AS, miR-26, miR-148, miR-28, let71, miR-191, miR-146, miR-422, miR-297, miR-128, miR-93, miR-105, and/or miR-25 in a blood sample enriched in CD34+ cells from an individual with AML harboring an abnormality of chromosome 7 (monosomy 7/deletion 7) can be used for a prognostic assay. Altered expression of miR-23, miR-150, miR-184, miR-100, miR-199a, miR-148, miR-105, miR-195, miR-341, miR-30a-AS, miR-199a-AS, miR-145, miR-205, miR-126, miR-221, miR-342, miR-335, miR-106, miR-16, miR-22, miR-139, miR-26, miR-223, miR-10a in a blood sample enriched in CD34+ cells from an individual with AML who harbors a relatively normal karyotype can be used for a prognostic assay.

Prognostic assays featuring any one or combination of the miRNAs listed above could be used to assess an AML patient to determine whether an aggressive treatment regimen is justified. As with the diagnostic assays mentioned above, the absolute values that define low expression will depend on the platform used to measure the miRNA(s). The same methods described for the diagnostic assays could be used for a prognostic assays.

### I. MIRNA MOLECULES

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease III-like nuclease enzyme called Dicer. The processed miRNA is typically a portion of the stem.

The processed miRNA (also referred to as “mature miRNA”) become part of a large complex to down-regulate a particular target gene. Examples of animal miRNAs include those that imperfectly basepair with the target, which halts translation (Olsen et al., 1999; Seggerson et al., 2002). siRNA molecules also are processed by Dicer, but from a long, double-stranded RNA molecule. siRNAs are not naturally found in animal cells, but they can direct the sequence-specific cleavage of an mRNA target through a RNA-induced silencing complex (RISC) (Denli et al., 2003).

### A. Array Preparation

The present invention concerns the preparation and use of miRNA arrays or miRNA probe arrays, which are macroarrays or microarrays of nucleic acid molecules (probes) that are fully or nearly complementary or identical to a plurality of miRNA molecules or precursor miRNA molecules positioned on a support or support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass, metal, plastic, latex, and silicon. Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect miRNA; consequently, methods and compositions may be used with a variety of different types of miRNA arrays.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 2, 20, 25, 50, 80, 100 or more different probes. It is contemplated that they may contain 1000, 16,000, 65,000, 250,000 or 1,000,000 or more different probes. The probes can be directed to targets in one or more different organisms or cell types. The oligonucleotide probes range from 5 to 50, 5 to 45, 10 to 40, 9 to 34, or 15 to 40 nucleotides in length in some embodiments. In certain embodiments, the oligonucleotide probes are 5, 10, 15, 20 to 20, 25, 30, 35, 40 nucleotides in length including all integers and ranges there between.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### B. Sample Preparation

It is contemplated that the miRNA of a wide variety of samples can be analyzed using the arrays, index of miRNA probes, or array technology of the invention. While endogenous miRNA is contemplated for use with compositions and methods of the invention, recombinant miRNA—including nucleic acids that are complementary or identical to endogenous miRNA or precursor miRNA—can also be handled and analyzed as described herein. Samples may be biological samples, in which case, they can be from biopsy, fine needle aspirates, exfoliates, blood, tissue, organs, semen, saliva, tears, other bodily fluid, hair follicles, skin, or any sample containing or constituting biological cells, particularly CD+ cells. In certain embodiments, samples may be, but are not limited to, blood, or cells purified or enriched to some extent from blood or other bodily fluids or tissues. Alternatively, the sample may not be a biological sample, but be a chemical mixture, such as a cell-free reaction mixture (which may contain one or more biological enzymes).

### C. Hybridization

After an array or a set of miRNA probes is prepared and/or the miRNA in the sample or miRNA probe is labeled, the population of target nucleic acids is contacted with the array or probes under hybridization conditions, where such conditions can be adjusted, as desired, to provide for an optimum level of specificity in view of the particular assay being performed. Suitable hybridization conditions are well known to those of skill in the art and reviewed in Sambrook et al. (2001) and WO 95/21944. Of particular interest in many embodiments is the use of stringent conditions during hybridization. Stringent conditions are known to those of skill in the art.

It is specifically contemplated that a single array or set of probes may be contacted with multiple samples. The samples may be labeled with different labels to distinguish the samples. For example, a single array can be contacted with a tumor tissue sample labeled with Cy3, and normal tissue sample labeled with Cy5. Differences between the samples for particular miRNAs corresponding to probes on the array can be readily ascertained and quantified.

The small surface area of the array permits uniform hybridization conditions, such as temperature regulation and salt content. Moreover, because of the small area occupied by the high density arrays, hybridization may be carried out in extremely small fluid volumes (e.g., about 250 μl or less, including volumes of about or less than about 5, 10, 25, 50, 60, 70, 80, 90, 100 μl, or any range derivable therein). In small volumes, hybridization may proceed very rapidly.

### D. Differential Expression Analyses

Arrays of the invention can be used to detect differences between two samples. Specifically contemplated applications include identifying and/or quantifying differences between miRNA from a sample that is normal and from a sample that is not normal, between a cancerous condition and a non-cancerous condition, or between two differently treated samples. Also, miRNA may be compared between a sample believed to be susceptible to a particular disease or condition and one believed to be not susceptible or resistant to that disease or condition. A sample that is not normal is one exhibiting phenotypic or genotypic trait(s) of a disease or condition, or one believed to be not normal with respect to that disease or condition. It may be compared to a cell that is normal with respect to that disease or condition. Phenotypic traits include symptoms of, or susceptibility to, a disease or condition of which a component is or may or may not be genetic, or caused by a hyperproliferative or neoplastic cell or cells.

An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., (1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes. Arrays may be packaged in such a manner as to allow for diagnostics or other manipulation of an all inclusive device, see for example, U.S. Pat. Nos. 5,856,174 and 5,922,591 incorporated in their entirety by reference for all purposes. See also U.S. patent application Ser. No. 09/545,207, filed Apr. 7, 2000 for additional information concerning arrays, their manufacture, and their characteristics, which is incorporated by reference in its entirety for all purposes.

Particularly, arrays can be used to evaluate samples with respect to AML and related conditions. It is specifically contemplated that the invention can be used to evaluate differences between stages or sub-classifications of disease, such as between monosomy 7 AML, inv (16) AML, or cytogenetically normal AML.

Phenotypic traits to be assessed include characteristics such as longevity, morbidity, expected survival, susceptibility or receptivity to particular drugs or therapeutic treatments (drug efficacy), and risk of drug toxicity. Samples that differ in these phenotypic traits may also be evaluated using the compositions and methods described.

In certain embodiments, miRNA profiles may be generated to evaluate and correlate those profiles with pharmacokinetics. For example, miRNA profiles may be created and evaluated for patient tumor and blood samples prior to the patient's being treated or during treatment to determine if there are miRNAs whose expression correlates with the outcome of the patient's treatment. Identification of differential miRNAs can lead to a diagnostic assay for evaluation of tumor and/or blood samples to determine what drug regimen the patient should be provided. In addition, it can be used to identify or select patients suitable for a particular clinical trial. If a miRNA profile is determined to be correlated with drug efficacy or drug toxicity that may be relevant to whether that patient is an appropriate patient for receiving the drug or for a particular dosage of the drug.

In addition to the above prognostic assay, blood samples from patients with a variety of diseases can be evaluated to determine if different diseases can be identified based on blood miRNA levels. A diagnostic assay can be created based on the profiles that doctors can use to identify individuals with a disease or who are at risk to develop a disease. Alternatively, treatments can be designed based on miRNA profiling. Examples of such methods and compositions are described in the U.S. Provisional Patent Application entitled “Methods and Compositions Involving miRNA and miRNA Inhibitor Molecules” filed on May 23, 2005 in the names of David Brown, Lance Ford, Angie Cheng and Rich Jarvis, which is hereby incorporated by reference in its entirety.

### E. Other Assays

In addition to the use of arrays and microarrays, it is contemplated that a number of difference assays could be employed to analyze miRNAs, their activities, and their effects. Such assays include, but are not limited to, nucleic amplification, polymerase chain reaction, quantitative PCR, RT-PCR, in situ hybridization, Northern hybridization, hybridization protection assay (HPA) (GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

### II. NUCLEIC ACIDS

The present invention concerns miRNAs that can be labeled, used in array analysis, or employed in diagnostic, therapeutic, or prognostic applications, particularly those related to pathological conditions such as cancer and in particular AML. The RNA may have been endogenously produced by a cell, or been synthesized or produced chemically or recombinantly. They may be isolated and/or purified. Table 1 indicates which SEQ ID NO correspond to the mature sequence. The name of the miRNA is often abbreviated and referred to without a hsa-prefix and will be understood as such, depending on the context. Unless otherwise indicated, miRNAs referred to in the application are human sequences identified as miR-X or let-X, where X is a number and/or letter.

In certain aspects, a miRNA probe designated by a suffix “5P” or “3P” can be used. “5P” indicates that the mature miRNA derives from the 5′ end of the precursor and a corresponding “3P” indicates that it derives from the 3′ end of the precursor, as described on the World Wide Web at sanger.ac.uk. Moreover, in some embodiments, a miRNA probe is used that does not correspond to a known human miRNA. It is contemplated that these non-human miRNA probes may be used in embodiments of the invention or that there may exist a human miRNA that is homologous to the non-human miRNA. In other embodiments, any mammalian cell, biological sample, or preparation thereof may be employed.

In some embodiments of the invention, methods and compositions involving miRNA may concern miRNA and/or other nucleic acids. Nucleic acids may be, be at least, or be at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length. Such lengths cover the lengths of processed miRNA, miRNA probes, precursor miRNA, miRNA containing vectors, control nucleic acids, and other probes and primers. In many embodiments, miRNA are 19-24 nucleotides in length, while miRNA probes are 19-35 nucleotides in length, depending on the length of the processed miRNA and any flanking regions added. miRNA precursors are generally between 62 and 110 nucleotides in humans.

Nucleic acids of the invention may have regions of identity or complementarity to another nucleic acid. It is contemplated that the region of complementarity or identity can be at least 5 contiguous residues, though it is specifically contemplated that the region is, is at least, or is at most 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides. It is further understood that the length of complementarity within a precursor miRNA or between a miRNA probe and a miRNA or a miRNA gene are such lengths. Moreover, the complementarity may be expressed as a percentage, meaning that the complementarity between a probe and its target is 90% or greater over the length of the probe. In some embodiments, complementarity is or is at least 90%, 95% or 100%. In particular, such lengths may be applied to any nucleic acid comprising a nucleic acid sequence identified in any of SEQ ID NO:1 through SEQ ID NO:62 or any other sequence disclosed herein. Typically, the commonly used name of the miRNA is given (with its identifying source in the prefix, for example, “hsa” for human sequences) and the processed miRNA sequence. Unless otherwise indicated, a miRNA without a prefix will be understood to refer to a human miRNA. Moreover, a lowercase letter in a miRNA name may or may not be lowercase; for example, hsa-mir-130b can also be referred to as miR-130B. The term “miRNA probe” refers to a nucleic acid probe that can identify a particular miRNA or structurally related miRNAs.

It is understood that some miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The term “recombinant” may be used and this generally refers to a molecule that has been manipulated in vitro or that is a replicated or expressed product of such a molecule.

The term “nucleic acid” is well known in the art. A “nucleic acid” as used herein will generally refer to a molecule (one or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence. For example, precursor miRNA may have a self-complementary region, which is up to 100% complementary. miRNA probes or nucleic acids of the invention can include, can be or can be at least 60, 65, 70, 75, 80, 85, 90, 95, 96, 97, 98, 99 or 100% complementary to their target.

It is understood that a “synthetic nucleic acid” of the invention means that the nucleic acid does not have a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

While embodiments of the invention may involve synthetic miRNAs or synthetic nucleic acids, in some embodiments of the invention, the nucleic acid molecule(s) need not be “synthetic.” In certain embodiments, a non-synthetic miRNA employed in methods and compositions of the invention may have the entire sequence and structure of a naturally occurring miRNA precursor or the mature miRNA. For example, non-synthetic miRNAs used in methods and compositions of the invention may not have one or more modified nucleotides or nucleotide analogs. In these embodiments, the non-synthetic miRNA may or may not be recombinantly produced. In particular embodiments, the nucleic acid in methods and/or compositions of the invention is specifically a synthetic miRNA and not a non-synthetic miRNA (that is, not a miRNA that qualifies as “synthetic”); though in other embodiments, the invention specifically involves a non-synthetic miRNA and not a synthetic miRNA. Any embodiments discussed with respect to the use of synthetic miRNAs can be applied with respect to non-synthetic miRNAs, and vice versa.

It will be understood that the term “naturally occurring” refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA.” Corresponding miRNA sequences that can be used in the context of the invention include, but are not limited to, all or a portion of those sequences in SEQ ID NOs: 1-62, as well as any other miRNA sequence, miRNA precursor sequence, or any sequence complementary thereof. In some embodiments, the sequence is or is derived from or contains all or part of a sequence identified in Table 1 to target a particular miRNA (or set of miRNAs) that can be used with that sequence.

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

### A. Nucleobase, Nucleoside, Nucleotide, and Modified Nucleotides

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. Other examples are well known to those of skill in the art.

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring. Different types of covalent attachment(s) of a nucleobase to a nucleobase linker moiety are known in the art (Kornberg and Baker, 1992).

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

A nucleic acid may comprise, or be composed entirely of, a derivative or analog of a nucleobase, a nucleobase linker moiety and/or backbone moiety that may be present in a naturally occurring nucleic acid. RNA with nucleic acid analogs may also be labeled according to methods of the invention. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids include those in: U.S. Pat. Nos. 5,681,947, 5,652,099 and 5,763,167, 5,614,617, 5,670,663, 5,872,232, 5,859,221, 5,446,137, 5,886,165, 5,714,606, 5,672,697, 5,466,786, 5,792,847, 5,223,618, 5,470,967, 5,378,825, 5,777,092, 5,623,070, 5,610,289, 5,602,240, 5,858,988, 5,214,136, 5,700,922, 5,708,154, 5,728,525, 5,637,683, 6,251,666, 5,480,980, and 5,728,525, each of which is incorporated herein by reference in its entirety.

Labeling methods and kits of the invention specifically contemplate the use of nucleotides that are both modified for attachment of a label and can be incorporated into a miRNA molecule. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Modified nucleotides for use in the invention are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, Biosearch Technologies and NEN. Functional groups may be prepared according to ways known to those of skill in the art, including the representative information found in U.S. Pat. Nos. 4,404,289; 4,405,711; 4,337,063 and 5,268,486, and U.K. Patent 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N6-(4-amino)butyl-ATP, N6-(6-amino)butyl-ATP, N4-[2,2-oxy-bis-(ethylamine)]-CTP; N6-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N6-(4-amino)butyl-dATP, N6-(6-amino)butyl-dATP, N4-[2,2-oxy-bis-(ethylamine)]-dCTP; N6-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

### B. Preparation of Nucleic Acids

A nucleic acid may be made by any technique known to one of ordinary skill in the art, such as for example, chemical synthesis, enzymatic production or biological production. It is specifically contemplated that miRNA probes of the invention are chemically synthesized.

In some embodiments of the invention, miRNAs are recovered or isolated from a biological sample. The miRNA may be recombinant or it may be natural or endogenous to the cell (produced from the cell's genome). It is contemplated that a biological sample may be treated in a way so as to enhance the recovery of small RNA molecules such as miRNA. U.S. patent application Ser. No. 10/667,126 describes such methods and it is specifically incorporated by reference herein. Generally, methods involve lysing cells with a solution having guanidinium and a detergent.

Alternatively, nucleic acid synthesis is performed according to standard methods. See, for example, Itakura and Riggs (1980) and U.S. Pat. Nos. 4,704,362, 5,221,619, and 5,583,013, each of which is incorporated herein by reference. Non-limiting examples of a synthetic nucleic acid (e.g., a synthetic oligonucleotide), include a nucleic acid made by in vitro chemically synthesis using phosphotriester, phosphite, or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR (see for example, U.S. Pat. Nos. 4,683,202 and 4,682,195, each incorporated herein by reference), or the synthesis of an oligonucleotide described in U.S. Pat. No. 5,645,897, incorporated herein by reference. See also Sambrook et al., 2001, incorporated herein by reference).

Oligonucleotide synthesis is well known to those of skill in the art. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Recombinant methods for producing nucleic acids in a cell are well known to those of skill in the art. These include the use of vectors (viral and non-viral), plasmids, cosmids, and other vehicles for delivering a nucleic acid to a cell, which may be the target cell (e.g., a cancer cell) or simply a host cell (to produce large quantities of the desired RNA molecule). Alternatively, such vehicles can be used in the context of a cell free system so long as the reagents for generating the RNA molecule are present. Such methods include those described in Sambrook, 2003, Sambrook, 2001 and Sambrook, 1989, which are hereby incorporated by reference.

### C. Isolation of Nucleic Acids

Nucleic acids may be isolated using techniques well known to those of skill in the art, though in particular embodiments, methods for isolating small nucleic acid molecules, and/or isolating RNA molecules can be employed. Chromatography is a process often used to separate or isolate nucleic acids from protein or from other nucleic acids. Such methods can involve electrophoresis with a gel matrix, filter columns, alcohol precipitation, and/or other chromatography. If miRNA from cells is to be used or evaluated, methods generally involve lysing the cells with a chaotropic (e.g., guanidinium isothiocyanate) and/or detergent (e.g., N-lauroyl sarcosine) prior to implementing processes for isolating particular populations of RNA.

In particular methods for separating miRNA from other nucleic acids, a gel matrix is prepared using polyacrylamide, though agarose can also be used. The gels may be graded by concentration or they may be uniform. Plates or tubing can be used to hold the gel matrix for electrophoresis. Usually one-dimensional electrophoresis is employed for the separation of nucleic acids. Plates are used to prepare a slab gel, while the tubing (glass or rubber, typically) can be used to prepare a tube gel. The phrase “tube electrophoresis” refers to the use of a tube or tubing, instead of plates, to form the gel. Materials for implementing tube electrophoresis can be readily prepared by a person of skill in the art or purchased, such as from C.B.S. Scientific Co., Inc. or Scie-Plas.

Methods may involve the use of organic solvents and/or alcohol to isolate nucleic acids, particularly miRNA used in methods and compositions of the invention. Some embodiments are described in U.S. patent application Ser. No. 10/667,126, which is hereby incorporated by reference. Generally, this disclosure provides methods for efficiently isolating small RNA molecules from cells comprising: adding an alcohol solution to a cell lysate and applying the alcohol/lysate mixture to a solid support before eluting the RNA molecules from the solid support. In some embodiments, the amount of alcohol added to a cell lysate achieves an alcohol concentration of about 55% to 60%. While different alcohols can be employed, ethanol works well. A solid support may be any structure, and it includes beads, filters, and columns, which may include a mineral or polymer support with electronegative groups. A glass fiber filter or column has worked particularly well for such isolation procedures.

In specific embodiments, miRNA isolation processes include: a) lysing cells in the sample with a lysing solution comprising guanidinium, wherein a lysate with a concentration of at least about 1 M guanidinium is produced; b) extracting miRNA molecules from the lysate with an extraction solution comprising phenol; c) adding to the lysate an alcohol solution for form a lysate/alcohol mixture, wherein the concentration of alcohol in the mixture is between about 35% to about 70%; d) applying the lysate/alcohol mixture to a solid support; e) eluting the miRNA molecules from the solid support with an ionic solution; and, f) capturing the miRNA molecules. Typically the sample is dried down and resuspended in a liquid and volume appropriate for subsequent manipulation.

### III. LABELS AND LABELING TECHNIQUES

In some embodiments, the present invention concerns miRNA that are labeled. It is contemplated that miRNA may first be isolated and/or purified prior to labeling. This may achieve a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

### A. Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to a miRNA, and the unlabeled nucleotide is modified with a chemical moiety that enables it to be subsequently labeled. In embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNA is how to label the already existing molecule. The present invention concerns the use of an enzyme capable of using a di- or tri-phosphate ribonucleotide or deoxyribonucleotide as a substrate for its addition to a miRNA. Moreover, in specific embodiments, it involves using a modified di- or tri-phosphate ribonucleotide, which is added to the 3′ end of a miRNA. Enzymes capable of adding such nucleotides include, but are not limited to, poly(A) polymerase, terminal transferase, and polynucleotide phosphorylase. In specific embodiments of the invention, a ligase is contemplated as not being the enzyme used to add the label, and instead, a non-ligase enzyme is employed. Terminal transferase catalyzes the addition of nucleotides to the 3′ terminus of a nucleic acid. Polynucleotide phosphorylase can polymerize nucleotide diphosphates without the need for a primer.

### B. Labels

Labels on miRNA or miRNA probes may be colorimetric (includes visible and UV spectrum, including fluorescent), luminescent, enzymatic, or positron emitting (including radioactive). The label may be detected directly or indirectly. Radioactive labels include ¹²⁵I, ³²P, ³³P, and ³⁵S. Examples of enzymatic labels include alkaline phosphatase, luciferase, horseradish peroxidase, and P-galactosidase. Labels can also be proteins with luminescent properties, e.g., green fluorescent protein and phicoerythrin.

The colorimetric and fluorescent labels contemplated for use as conjugates include, but are not limited to, Alexa Fluor dyes, BODIPY dyes, such as BODIPY FL; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and, TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP.

Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP.

It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference).

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

### C. Visualization Techniques

A number of techniques for visualizing or detecting labeled nucleic acids are readily available. Such techniques include, microscopy, arrays, Fluorometry, Light cyclers or other real time PCR machines, FACS analysis, scintillation counters, Phosphoimagers, Geiger counters, MRI, CAT, antibody-based detection methods (Westerns, immunofluorescence, immunohistochemistry), histochemical techniques, HPLC (Griffey et al., 1997), spectroscopy, capillary gel electrophoresis (Cummins et al., 1996), spectroscopy; mass spectroscopy; radiological techniques; and mass balance techniques.

When two or more differentially colored labels are employed, fluorescent resonance energy transfer (FRET) techniques may be employed to characterize association of one or more nucleic acid. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule.

### IV. KITS

Any of the compositions described herein may be comprised in a kit. In a non-limiting example, reagents for isolating miRNA, labeling miRNA, and/or evaluating a miRNA population using an array, nucleic acid amplification, and/or hybridization can be included in a kit, as well reagents for preparation of samples from blood samples. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. In certain aspects, the kit can include amplification reagents. In other aspects, the kit may include various supports, such as glass, nylon, polymeric beads, and the like, and/or reagents for coupling any probes and/or target nucleic acids. It may also include one or more buffers, such as reaction buffer, labeling buffer, washing buffer, or a hybridization buffer, compounds for preparing the miRNA probes, and components for isolating miRNA. Other kits of the invention may include components for making a nucleic acid array comprising miRNA, and thus, may include, for example, a solid support.

Kits for implementing methods of the invention described herein are specifically contemplated. In some embodiments, there are kits for preparing miRNA for multi-labeling and kits for preparing miRNA probes and/or miRNA arrays. In these embodiments, kit comprise, in suitable container means, 1, 2, 3, 4, 5, 6, 7, 8, 9, 10, 11, 12 or more of the following: (1) poly(A) polymerase; (2) unmodified nucleotides (G, A, T, C, and/or U); (3) a modified nucleotide (labeled or unlabeled); (4) poly(A) polymerase buffer; and, (5) at least one microfilter; (6) label that can be attached to a nucleotide; (7) at least one miRNA probe; (8) reaction buffer; (9) a miRNA array or components for making such an array; (10) acetic acid; (11) alcohol; (12) solutions for preparing, isolating, enriching, and purifying miRNAs or miRNA probes or arrays. Other reagents include those generally used for manipulating RNA, such as formamide, loading dye, ribonuclease inhibitors, and DNase.

In specific embodiments, kits of the invention include an array containing miRNA probes, as described in the application. An array may have probes corresponding to all known miRNAs of an organism or a particular tissue or organ in particular conditions, or to a subset of such probes. The subset of probes on arrays of the invention may be or include those identified as relevant to a particular diagnostic, therapeutic, or prognostic application. For example, the array may contain one or more probes that is indicative or suggestive of (1) a disease or condition (acute myeloid leukemia), (2) susceptibility or resistance to a particular drug or treatment; (3) susceptibility to toxicity from a drug or substance; (4) the stage of development or severity of a disease or condition (prognosis); and (5) genetic predisposition to a disease or condition.

For any kit embodiment, including an array, there can be nucleic acid molecules that contain or can be used to amplify a sequence that is a variant of, identical to or complementary to all or part of any of SEQ ID NOS: 1-62. In certain embodiments, a kit or array of the invention can contain one or more probes for the miRNAs identified by SEQ ID NOS:1-62. Any nucleic acid discussed above may be implemented as part of a kit.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits of the invention. The dye may then be resuspended in any suitable solvent, such as DMSO.

Such kits may also include components that facilitate isolation of the labeled miRNA. It may also include components that preserve or maintain the miRNA or that protect against its degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

Kits of the invention may also include one or more of the following: Control RNA; nuclease-free water; RNase-free containers, such as 1.5 ml tubes; RNase-free elution tubes; PEG or dextran; ethanol; acetic acid; sodium acetate; ammonium acetate; guanidinium; detergent; nucleic acid size marker; RNase-free tube tips; and RNase or DNase inhibitors.

It is contemplated that such reagents are embodiments of kits of the invention. Such kits, however, are not limited to the particular items identified above and may include any reagent used for the manipulation or characterization of miRNA.

### V. THERAPEUTIC METHODS

Embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors of miRNA in a cell. The term “short” refers to a length of a single polynucleotide that is 25, 50, 100, or 150 nucleotides or fewer, including all integers or range derivable there between. The nucleic acid molecules are typically synthetic. The term “synthetic” means the nucleic acid molecule is isolated and not identical in sequence (the entire sequence) and/or chemical structure to a naturally-occurring nucleic acid molecule, such as an endogenous precursor miRNA or miRNA molecule. While in some embodiments, nucleic acids of the invention do not have an entire sequence that is identical to a sequence of a naturally-occurring nucleic acid, such molecules may encompass all or part of a naturally-occurring sequence. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together. In certain aspects, synthetic miRNA of the invention are RNA or RNA analogs. miRNA inhibitors may be DNA or RNA, or analogs thereof. miRNA and miRNA inhibitors of the invention are collectively referred to as “synthetic nucleic acids.”

In some embodiments, there is a synthetic miRNA having a length of between 17 and 130 residues. The present invention concerns synthetic miRNA molecules that are, are at least, or are at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 140, 145, 150, 160, 170, 180, 190, 200 or more residues in length, including any integer or any range derivable therein.

In certain embodiments, synthetic miRNA have (a) a “miRNA region” whose sequence from 5′ to 3′ is identical to all or a segment of a mature miRNA sequence, and (b) a “complementary region” whose sequence from 5′ to 3′ is between 60% and 100% complementary to the miRNA sequence. In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA.

The term “complementary region” refers to a region of a synthetic miRNA that is or is at least 60% complementary to the mature, naturally occurring miRNA sequence that the miRNA region is identical to. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, an miRNA inhibitor has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. Probe sequences for miRNAs are disclosed in Table 1. One of skill in the art could use a portion of the probe sequence that is complementary to the sequence of a mature miRNA as the sequence for a miRNA inhibitor. Moreover, that portion of the probe sequence can be altered so that it is still 90% complementary to the sequence of a mature miRNA.

In some embodiments, of the invention, a synthetic miRNA contains one or more design elements. These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2′O-Me (2′oxygen-methyl), DMTO (4,4′-dimethoxytrityl with oxygen), fluoroscein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there are one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification. In further embodiments, there are one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

Methods of the invention include reducing or eliminating activity of one or more miRNAs in a cell comprising introducing into a cell a miRNA inhibitor; or supplying or enhancing the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or a miRNA inhibitor are synthetic, as discussed above.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced miRNA. It is contemplated, however, that the miRNA molecule introduced into a cell is not a mature miRNA but is capable of becoming a mature miRNA under the appropriate physiological conditions. In cases in which a particular corresponding miRNA is being inhibited by a miRNA inhibitor, the particular miRNA will be referred to as the targeted miRNA. It is contemplated that multiple corresponding miRNAs may be involved. In particular embodiments, more than one miRNA molecule is introduced into a cell. Moreover, in other embodiments, more than one miRNA inhibitor is introduced into a cell. Furthermore, a combination of miRNA molecule(s) and miRNA inhibitor(s) may be introduced into a cell.

Methods include identifying a cell or patient in need of inducing those cellular characteristics. Also, it will be understood that an amount of a synthetic nucleic acid that is provided to a cell or organism is an “effective amount,” which refers to an amount needed to achieve a desired goal, such as inducing a particular cellular characteristic(s).

In certain embodiments of the methods include providing or introducing to a cell a nucleic acid molecule corresponding to a mature miRNA in the cell in an amount effective to achieve a desired physiological result.

Moreover, methods can involve providing synthetic or nonsynthetic miRNA molecules. It is contemplated that in these embodiments, methods may or may not be limited to providing only one or more synthetic miRNA molecules or only on or more nonsynthetic miRNA molecules. Thus, in certain embodiments, methods may involve providing both synthetic and nonsynthetic miRNA molecules. In this situation, a cell or cells are most likely provided a synthetic miRNA molecule corresponding to a particular miRNA and a nonsynthetic miRNA molecule corresponding to a different miRNA. Furthermore, any method articulated a list of miRNAs using Markush group language may be articulated without the Markush group language and a disjunctive article (i.e., or) instead, and vice versa.

In some embodiments, there is a method for reducing or inhibiting cell proliferation in a cell comprising introducing into or providing to the cell an effective amount of (i) a miRNA inhibitor molecule or (ii) a synthetic or nonsynthetic miRNA molecule that corresponds to a miRNA sequence. In certain embodiments the methods involves introducing into the cell an effective amount of (i) an miRNA inhibitor molecule having a 5′ to 3′ sequence that is at least 90% complementary to the 5′ to 3′ sequence of one or more mature miRNA of Table 1.

Certain embodiments of the invention include methods of treating a leukemic, in particular an acute myeloid leukemic condition. In one aspect, the method comprises contacting a leukemic cell with one or more nucleic acid, synthetic miRNA, or miRNA comprising at least one nucleic acid segment having all or a portion of a miRNA sequence. The segment may be 5, 6, 7, 8, 9, 10, 11, 12, 13, 14, 15, 16, 17, 18, 19, 20, 21, 22, 23, 24, 25, 30 or more nucleotides or nucleotide analog, including all integers there between. An aspect of the invention includes the modulation of a miRNA or a mRNA within a target cell, such as a leukemic cell.

Typically, an endogenous gene, miRNA or mRNA is modulated in the cell. In particular embodiments, the nucleic acid sequence comprises at least one segment that is at least 70, 75, 80, 85, 90, 95, or 100% identical in nucleic acid sequence to one or more miRNA sequence listed in Table 1. Modulation of the expression or processing of an endogenous gene, miRNA, or mRNA can be through modulation of the processing of a mRNA, such processing including transcription, transportation and/or translation with in a cell. Modulation may also be effected by the inhibition or enhancement of miRNA activity with a cell, tissue, or organ. Such processing may effect the expression of an encoded product or the stability of the mRNA. In still other embodiments, a nucleic acid sequence can comprise a modified nucleic acid sequence. In certain aspects, one or more miRNA sequence may include or comprise a modified nucleobase or nucleic acid sequence.

It will be understood in methods of the invention that a cell or other biological matter such as an organism (including patients) can be provided a miRNA or miRNA molecule corresponding to a particular miRNA by administering to the cell or organism a nucleic acid molecule that functions as the corresponding miRNA once inside the cell. The form of the molecule provided to the cell may not be the form that acts a miRNA once inside the cell. Thus, it is contemplated that in some embodiments, biological matter is provided a synthetic miRNA or a nonsynthetic miRNA, such as one that becomes processed into a mature and active miRNA once it has access to the cell's miRNA processing machinery. In certain embodiments, it is specifically contemplated that the miRNA molecule provided to the biological matter is not a mature miRNA molecule but a nucleic acid molecule that can be processed into the mature miRNA once it is accessible to miRNA processing machinery. The term “nonsynthetic” in the context of miRNA means that the miRNA is not “synthetic,” as defined herein. Furthermore, it is contemplated that in embodiments of the invention that concern the use of synthetic miRNAs, the use of corresponding nonsynthetic miRNAs is also considered an aspect of the invention, and vice versa. It will be understand that the term “providing” an agent is used to include “administering” the agent to a patient.

In certain embodiments, methods also include targeting a miRNA to modulate in a cell or organism. The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In some embodiments, the miRNA targeted to be modulated is a miRNA that affects a disease, condition, or pathway. In certain embodiments, the miRNA is targeted because a treatment can be provided by negative modulation of the targeted miRNA. In other embodiments, the miRNA is targeted because a treatment can be provided by positive modulation of the targeted miRNA.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability). Consequently, in some methods of the invention there is a step of identifying a patient in need of treatment that can be provided by the miRNA modulator(s). It is contemplated that an effective amount of a miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, a decrease in a symptom. For example, with respect to cancer, it is contemplated that a therapeutic benefit can be inhibition of tumor growth, prevention of metastasis, reduction in number of metastases, inhibition of cancer cell proliferation, inhibition of cancer cell proliferation, induction of cell death in cancer cells, inhibition of angiogenesis near cancer cells, induction of apoptosis of cancer cells, reduction in pain, reduction in risk of recurrence, induction of chemo- or radiosensitivity in cancer cells, prolongation of life, and/or delay of death directly or indirectly related to cancer.

Furthermore, it is contemplated that the miRNA compositions may be provided as part of a therapy to a patient, in conjunction with traditional therapies or preventative agents. Moreover, it is contemplated that any method discussed in the context of therapy may be applied as preventatively, particularly in a patient identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed. In certain embodiments, the therapeutic drug is a cancer therapeutic. Consequently, in some embodiments, there is a method of treating cancer in a patient comprising administering to the patient the cancer therapeutic and an effective amount of at least one miRNA molecule that improves the efficacy of the cancer therapeutic or protects non-cancer cells. Cancer therapies also include a variety of combination therapies with both chemical and radiation based treatments. Combination chemotherapies include but are not limited to, for example, bevacizumab, cisplatin (CDDP), carboplatin, EGFR inhibitors (gefitinib and cetuximab), procarbazine, mechlorethamine, cyclophosphamide, camptothecin, COX-2 inhibitors (e.g., celecoxib) ifosfamide, melphalan, chlorambucil, busulfan, nitrosurea, dactinomycin, daunorubicin, doxorubicin (adriamycin), bleomycin, plicomycin, mitomycin, etoposide (VP16), tamoxifen, raloxifene, estrogen receptor binding agents, taxol, taxotere, gemcitabien, navelbine, farnesyl-protein transferase inhibitors, transplatinum, 5-fluorouracil, vincristin, vinblastin and methotrexate, or any analog or derivative variant of the foregoing.

Generally, inhibitors of miRNAs can be given to achieve the opposite effect as compared to when nucleic acid molecules corresponding to the mature miRNA are given. Similarly, nucleic acid molecules corresponding to the mature miRNA can be given to achieve the opposite effect as compared to when inhibitors of the miRNA are given. For example, miRNA molecules that increase cell proliferation can be provided to cells to increase proliferation or inhibitors of such molecules can be provided to cells to decrease cell proliferation. The present invention contemplates these embodiments in the context of the different physiological effects observed with the different miRNA molecules and miRNA inhibitors disclosed herein. These include, but are not limited to, the following physiological effects: increase and decreasing cell proliferation, increasing or decreasing apoptosis, increasing transformation, increasing or decreasing cell viability, activating ERK, activating/inducing or inhibiting hTert, inhibit stimulation of Stat3, reduce or increase viable cell number, and increase or decrease number of cells at a particular phase of the cell cycle. Methods of the invention are generally contemplated to include providing or introducing one or more different nucleic acid molecules corresponding to one or more different miRNA molecules. It is contemplated that the following, at least the following, or at most the following number of different nucleic acid molecules may be provided or introduced: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein. This also applies to the number of different miRNA molecules that can be provided or introduced into a cell.

### VI. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art. Unless otherwise designated, catalog numbers refer to products available by that number from Ambion, Inc.®, The RNA Company.

### Example 1

### Patient Classification and Sample Preparation

Blood samples were obtained from each patient at the time of diagnosis. Blood from AML patients was withdrawn using standard methods. Blood from normal individuals was withdrawn from the bone marrow to increase the percentage of mononuclear (CD34+) cells. Survival data derive from monitoring patients following the date that they were diagnosed. Survival times are based on the number of months between the original diagnosis of AML and the date that a patient succumbed to the disease.

Chromosome analysis for all samples was performed on metaphase spreads from direct preparations, as well as from 24 hour and 48 hour cultures of bone marrow and/or peripheral blood samples. AML classification was done by routine cytogenetics using G-banding (−7/7q−) or PCR (inv (16)). Classification of normal karyotype patients was done as recently described for the AML-SHG96 study group (Schaich et al., 2001). In brief, the normal karyotype patients were assigned based on the absence of the following translocations (−5/del(5q), −7/7q−), hypodiploid karyotypes (besides 45, X, −Y or −X), inv(3q), abnl12p, abnl11q, +11, +13, +21, +22, t(6;9); t(9;22); t(9;11); t(3;3), t(8;21), t(8;21) or any combination of detectable translocations.

CD34+ blood cells were prepared by a standardized ficoll gradient separation procedure and cryopreserved in vials containing 5-20×10⁶ cells. RNA from CD34+-enriched blood samples was purified using the mirVana™ RNA Isolation Kit (Ambion) according to the manufacturer's recommendations. The resulting RNA was used for array analysis and/or qRT-PCR as described below.

Quantitative RT-PCR was used for quantitative detection of mature miRNA transcripts. 10-20 pg of miRNA were used in the assay. qRT-primers and PCR kits (Ambion) included those for hsa-mir-16, hsa-mir-150, hsa-mir-23a, hsa-mir-25, hsa-mir-223, and hsa-mir-335. Data were normalized to 5S rRNA that was amplified with a specific amplification kit (Ambion). Assay linearity was verified by dilution curve analysis, and assay specificity was evaluated by melting curve analysis. Relative gene expression was calculated using the previously described 2^{-ΔΔC}_{T} method (Livak and Schmittgen, 2001).

### Example 2

### MicroRNAs Diagnostic for AML Patients with Inv (16)

Purified miRNA from twelve patients with AML caused by inv (16) and from three normal patients was labeled using the mirVana™ miRNA Labeling Kit (Ambion) according to the manufacturer's instructions. Samples were labeled with Cy3 and co-hybridized to miRNA arrays with Cy5-labeled miRNAs from a mixture of spleen and thymus. miRNA arrays contained probes for 203 human miRNAs. Following hybridization, a GenePix 4000B Array Scanner (Axon) was used to measure fluorescence at each of the miRNA probe spots on the array. Each element was located and analyzed using the GenePix Pro 5.0 software package (Axon). Data were filtered for quality and significance using the Longhorn Array Database (Killion et al, 2003) according to several data quality standards, including minimum intensity and pixel consistency. All data used for analysis had a signal-to-noise ratio >5, an average sum intensity 50% higher than that of the negative control spots, and a regression ratio >0.5. Data were normalized globally per array such that the average log ratio was 0 after normalization.

The array data from each of the AML patient samples was compared to each of the normal patient samples using the Cy5 signal from the spleen/thymus RNA for normalization. Although the relative expression levels of miRNAs in samples from the inv (16) and normal patients were remarkably similar, hierarchical clustering (performed by average linkage using uncentered Pearson correlation and Log2 normalization with 203 miRNAs) grouped the samples into normal and leukemia groups. As noted in Table 2, seventeen miRNAs were significantly altered in samples from the inv (16) patients relative to samples from the normal patients. Hsa-miR-29, miR-126, miR-21, miR-99, miR-125, miR-16, miR-20, miR-10, miR-199a-AS, miR-23, let-7, miR-106, miR-181, miR-15, and miR-26 were expressed at significantly lower levels in samples from the inv (16) patients. Hsa-miR-223 and miR-425 were both present at significantly higher levels in the inv (16) patient samples than in the normal patient samples. Any one or combination of these 17 miRNAs could be used as diagnostic analytes to determine if an individual has the inv (16) form of AML.

TABLE 2
MicroRNAs with significantly altered expression in
patients with acute myeloid leukemia caused by inv
(16) as compared to expression in normal patients.

| miRNA | log₂ AML/Normal |
|---|---|
| hsa-miR-223 | 1.649333 |
| hsa-miR-425 | 0.576 |
| Hsa-miR-26 | −0.68633 |
| Hsa-miR-15 | −0.719 |
| hsa-miR-181 | −0.75633 |
| hsa-miR-106 | −0.76767 |
| hsa-miR-let7 | −0.83967 |
| Hsa-miR-23 | −0.84367 |
| hsa-miR-199a-AS | −0.8485 |
| Hsa-miR-10 | −0.85967 |
| Hsa-miR-20 | −0.89367 |
| Hsa-miR-16 | −0.93267 |
| hsa-miR-125 | −1.00367 |
| Hsa-miR-29 | −1.015 |
| Hsa-miR-99 | −1.045 |
| Hsa-miR-21 | −1.17133 |
| hsa-miR-126 | −1.86533 |

### Example 3

### MicroRNAs Diagnostic for AML Patients with Monosomy 7/Deletion 7

RNA from seventeen monosomy 7 and deletion 7 (7q−) patients and ten normal controls was labeled using the mirVana™ miRNA Labeling Kit (Ambion) according to the manufacturer's instructions. The samples were labeled with Cy3, co-hybridized with a Cy5-labeled mixture of spleen and thymus miRNA, and analyzed as described in Example 2. The signal for each miRNA, in each leukemia patient, was compared to each of the normal controls. As with the inv (16) in Example 2 above, hierarchical clustering (performed by average linkage using uncentered Pearson correlation and 10g2 normalization) grouped the samples into normal and leukemia groups. As noted in Table 3, the expression of many miRNAs was significantly altered in the monosomy 7/deletion 7 patient samples relative to the normal patient samples. Hsa-miR-181, miR-126, miR-17-5p, miR-20, miR-23, miR-221, miR-106, miR-19, miR-21, miR-92, miR-16, miR-222, miR-223, miR-27a, miR-24, miR-26, miR-191, miR-30, miR-103, let-7, miR-15, miR-107, miR-146, miR-30a-5p, miR-93, miR-29, and miR-106 were significantly elevated in the samples from the monosomy 7/deletion 7 patients and hsa-miR-494, miR-513, and miR-449 were present at significantly lower levels. Any one or combination of these 30 miRNAs could be used as diagnostic analytes to determine if an individual has the monosomy 7/deletion 7 form of AML.

Any one or combination of these miRNAs could be used as diagnostic analytes to determine if an individual has the monosomy 7/deletion 7 form of AML.

TABLE 3
MicroRNAs with significantly altered expression in patients
with acute myeloid leukemia caused by monosomy 7/deletion
7 as compared to expression in normal patients.

| miRNA | log₂ AML/Normal |
|---|---|
| hsa-miR-181a | 2.825696429 |
| hsa-miR-20 | 2.793720588 |
| hsa-miR-106 | 2.7100625 |
| hsa-miR-23 | 2.6395 |
| hsa-miR-221 | 2.509361905 |
| hsa-miR-17-5p | 2.500860294 |
| hsa-miR-19 | 2.363617647 |
| hsa-miR-21 | 2.182420168 |
| hsa-miR-92 | 2.180277311 |
| hsa-miR-16 | 2.165044118 |
| hsa-miR-222 | 1.9601875 |
| hsa-miR-223 | 1.9135 |
| hsa-miR-27a | 1.850419118 |
| hsa-miR-24 | 1.684316176 |
| hsa-miR-26 | 1.677161765 |
| hsa-miR-191 | 1.590011765 |
| hsa-miR-30c | 1.5384 |
| hsa-miR-126 | 1.456625 |
| hsa-miR-103 | 1.427838235 |
| hsa-let-7 | 1.39925 |
| hsa-miR-15 | 1.383036765 |
| hsa-miR-107 | 1.328392857 |
| hsa-miR-30a-5p | 1.301823529 |
| hsa-miR-146 | 1.169553571 |
| hsa-miR-93 | 0.957142857 |
| hsa-miR-29a | 0.951083333 |
| hsa-miR-106 | 0.9385 |
| hsa-miR-449 | −0.942757353 |
| hsa-miR-513 | −1.3095625 |
| hsa-miR-494 | −2.257470588 |

### Example 4

### MicroRNAs Diagnostic for AML Patients with Normal Karyotypes

RNA from twenty-one AML patients with normal karyotypes and five normal controls was labeled using the mirVana™ miRNA Labeling Kit (Ambion) according to the manufacturer's instructions. The samples were labeled with Cy3, co-hybridized with Cy5 labeled mixture of spleen and thymus miRNA, and analyzed as described in Examples 2 and 3 above. Hierarchical clustering grouped the samples into normal and leukemia groups. As shown in Table 4, sixteen miRNAs were significantly altered in the normal karyotype patients relative to the normal controls. Hsa-miR-223, miR-16, miR-23, miR-342, miR-17-5p, miR-15, miR-150, miR-103, miR-107, miR-106, and miR-20, were present at significantly lower levels in the samples from the normal karyotype patients and miR-205, miR-181, miR-125, miR-143, and miR-199a were present at significantly higher levels. Any one or combination of these sixteen miRNAs could be used as diagnostic analytes to determine if an individual has the normal karyotype form of AML.

TABLE 4
MicroRNAs with significantly altered expression in patients
with acute myeloid leukemia having a normal karyotype
as compared to expression in normal patients.

| miRNA | log₂ AML/Normal |
|---|---|
| hsa-miR-223 | −3.117511 |
| hsa-miR-16 | −2.0216 |
| hsa-miR-23 | −1.333611 |
| hsa-miR-342 | −1.051625 |
| hsa-miR-17-5p | −0.921225 |
| hsa-miR-15 | −0.884 |
| hsa-miR-150 | −0.874756 |
| hsa-miR-103 | −0.847525 |
| hsa-miR-107 | −0.753 |
| hsa-miR-106 | −0.6642 |
| hsa-miR-20 | −0.641125 |
| hsa-miR-205 | 0.5205778 |
| hsa-miR-181 | 0.5272222 |
| hsa-miR-125 | 0.5400667 |
| hsa-miR-143 | 0.57945 |
| hsa-miR-199a | 0.701325 |

### Example 5

### MicroRNAs Prognostic for AML Patients with Monosomy 7/Deletion 7

The array data described in Example 3 above were evaluated to identify miRNAs whose expression levels correlated with patient survival. Patients were categorized into two groups, those who succumbed to disease within two years of diagnosis and those who survived beyond two years following diagnosis. ANOVA was used to identify miRNAs with expression levels that were significantly different between the two patient groups. To verify the array data and to expand the range of samples being tested, quantitative RT-PCR was performed as described in Example 1. RNA from CD34+-enriched blood samples from 34 monosomy 7/deletion 7 patients was purified using the mirVana™ RNA Isolation Kit (Ambion) according to the manufacturer's recommendation. Purified miRNA (10-20 pg) was used in the assay. Table 5 lists the miRNAs with p-values less than 0.10. Any one or combination of thirteen miRNAs, including hsa-miR-199a-AS, miR-26, miR-422, miR-297, miR-128, miR-93, miR-105, miR-25, miR-148, miR-28, let-71, miR-191, and miR-146, can be used to predict the prognosis of a patient. A physician would be able to use this information to determine how aggressively to treat a patient.

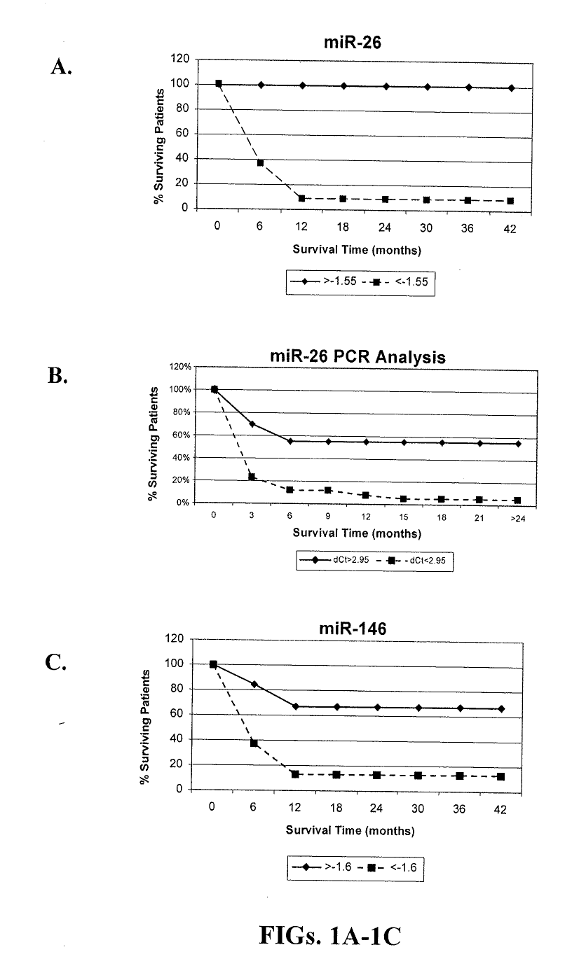

Survival curves (Kaplan-Meier) were generated for several of the miRNAs to show the distribution pattern of patient survival based on the classification of the patients using the expression levels of specific miRNAs. Survival curves generated from array data and PCR data for miR-26 and from array data for miR-146 are shown in FIGS. 1A-1C. As shown in FIGS. 1A and 1B, lower expression level of hsa-miR-26 is associated with reduced survival in these patients. This is supported by survival curves prepared from both array-generated and PCR-generated data (FIGS. 1A and 1B). Similarly, lower expression of miR-146 is associated with reduced survival in these patients (FIG. 1C).

TABLE 5
miRNAs with expression levels that differed significantly between
AML patients (monosomy 7/deletion 7) that succumbed to disease within
two years and patients that survived for over two years.

| miRNA | p-value |
|---|---|
| hsa-miR-199a-AS | 0.010914487 |
| hsa-miR-26 | 0.018494232 |
| hsa-miR-148 | 0.035714975 |
| hsa-miR-28 | 0.041801484 |
| hsa-let7i | 0.042802385 |
| hsa-miR-191 | 0.04863918 |
| hsa-miR-146 | 0.057407457 |
| hsa-miR-422 | 0.058678557 |
| hsa-miR-297 | 0.062197601 |
| hsa-miR-128 | 0.069114233 |
| hsa-miR-93 | 0.078840686 |
| hsa-miR-105 | 0.088985793 |
| hsa-miR-25 | 0.094606368 |

### Example 6

### miRNAs Prognostic for AML Patients with Normal Karyotypes

The array data described in Example 4 above were evaluated to identify miRNAs whose expression levels correlated with patient survival. Patients were categorized into two groups, those who succumbed to disease within two years of diagnosis and those who survived beyond two years following diagnosis. ANOVA was used to identify miRNAs with expression levels that were significantly different between the two patient groups. Table 6 below lists the miRNAs with p-values less than 0.10. Any one or combination of the following twenty-three miRNAs could be used to predict the prognosis of an AML patient with a normal karyotype: miR-23, miR-150, miR-184, miR-100, miR-199a, miR-148, miR-105, miR-195, miR-341, miR-30a-AS, miR-199a-AS, miR-145, miR-205, miR-126, miR-221, miR-342, miR-335, miR-106, miR-16, miR-22, miR-139, miR-26, miR-223, and miR-10a. A physician would be able to use this information to determine how aggressively to treat a patient.

TABLE 6
miRNAs with expression levels that differed significantly between
AML patients (normal karyotype) that succumbed to disease within
two years and patients that survived for over two years.

| miRNA | p-value |
|---|---|
| hsa-miR-150 | 0.001298 |
| hsa-miR-184 | 0.008248 |
| hsa-miR-100 | 0.010002 |
| hsa-miR-199a | 0.010694 |
| hsa-miR-148 | 0.01498 |
| hsa-miR-105 | 0.0136 |
| hsa-miR-195 | 0.015361 |
| hsa-miR-341 | 0.015513 |
| hsa-miR-30a-AS | 0.015543 |
| hsa-miR-199a-AS | 0.020759 |
| hsa-miR-145 | 0.021271 |
| hsa-miR-205 | 0.022004 |
| hsa-miR-126 | 0.024045 |
| hsa-miR-221 | 0.025098 |
| hsa-miR-342 | 0.035249 |
| hsa-miR-335 | 0.043165 |
| hsa-miR-106 | 0.044199 |
| hsa-miR-16 | 0.045647 |
| hsa-miR-22 | 0.047319 |
| hsa-miR-139 | 0.049123 |
| hsa-miR-26 | 0.057035 |
| hsa-miR-223 | 0.065507 |
| hsa-miR-10a | 0.072797 |
| Hsa-miR-23 | 0.02 |

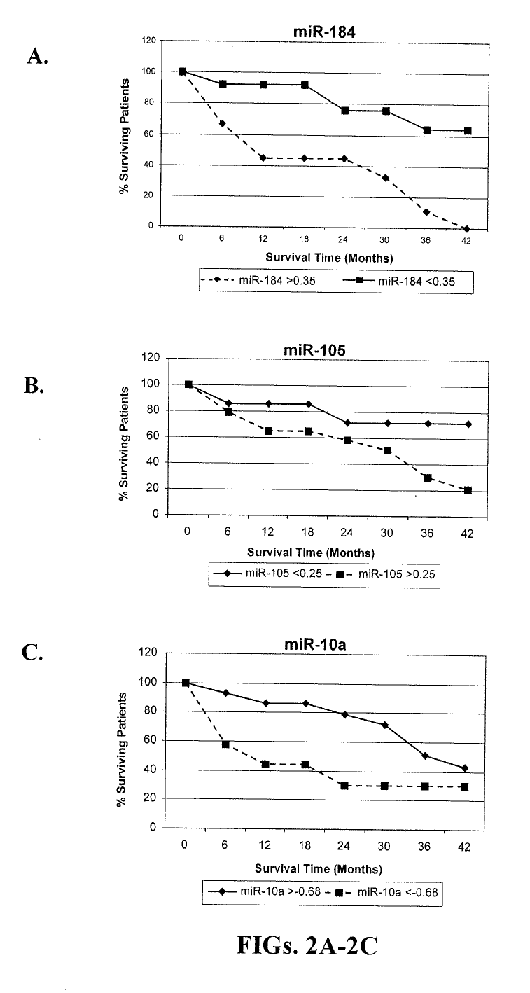

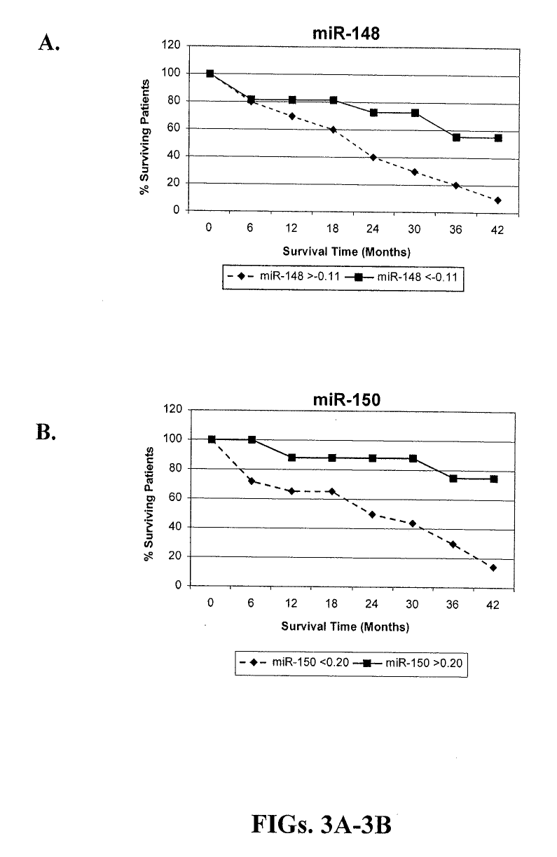

Survival curves (Kaplan-Meier) were generated for several of the miRNAs to show the distribution pattern of patient survival based on the classification of the patients using the expression levels of specific miRNAs. Survival curves are shown for miR-184, miR-105, and miR-10a in FIG. 2A-C and for miR-148 and miR-150 in FIG. 3A-B. Higher expression of miR-184, miR-105, or miR-148 is associated with reduced survival in this group of patients (FIGS. 2A and 2B, FIG. 3A). Lower expression of miR-10a or miR-150 is associated with reduced survival in this group of patients.

### REFERENCES

The following references, to the extent that they provide exemplary procedural or other details supplementary to those set forth herein, are specifically incorporated herein by reference.
U.S. Pat. No. 4,337,063
U.S. Pat. No. 4,404,289
U.S. Pat. No. 4,405,711
U.S. Pat. No. 4,659,774
U.S. Pat. No. 4,682,195
U.S. Pat. No. 4,683,202
U.S. Pat. No. 4,704,362
U.S. Pat. No. 4,816,571
U.S. Pat. No. 4,959,463
U.S. Pat. No. 5,141,813
U.S. Pat. No. 5,143,854
U.S. Pat. No. 5,202,231
U.S. Pat. No. 5,214,136
U.S. Pat. No. 5,221,619
U.S. Pat. No. 5,223,618
U.S. Pat. No. 5,242,974
U.S. Pat. No. 5,264,566
U.S. Pat. No. 5,268,486
U.S. Pat. No. 5,288,644
U.S. Pat. No. 5,324,633
U.S. Pat. No. 5,378,825
U.S. Pat. No. 5,384,261
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,470,967
U.S. Pat. No. 5,472,672
U.S. Pat. No. 5,480,980
U.S. Pat. No. 5,492,806
U.S. Pat. No. 5,503,980
U.S. Pat. No. 5,510,270
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,580,726
U.S. Pat. No. 5,580,732
U.S. Pat. No. 5,583,013
U.S. Pat. No. 5,593,839
U.S. Pat. No. 5,599,672
U.S. Pat. No. 5,599,695
U.S. Pat. No. 5,602,240
U.S. Pat. No. 5,602,244
U.S. Pat. No. 5,610,289
U.S. Pat. No. 5,610,287
U.S. Pat. No. 5,614,617
U.S. Pat. No. 5,623,070
U.S. Pat. No. 5,624,711
U.S. Pat. No. 5,631,134
U.S. Pat. No. 5,637,683
U.S. Pat. No. 5,639,603
U.S. Pat. No. 5,645,897
U.S. Pat. No. 5,652,099
U.S. Pat. No. 5,654,413
U.S. Pat. No. 5,658,734
U.S. Pat. No. 5,661,028
U.S. Pat. No. 5,665,547
U.S. Pat. No. 5,667,972
U.S. Pat. No. 5,670,663
U.S. Pat. No. 5,672,697
U.S. Pat. No. 5,677,195
U.S. Pat. No. 5,681,947
U.S. Pat. No. 5,695,940
U.S. Pat. No. 5,700,637
U.S. Pat. No. 5,700,922
U.S. Pat. No. 5,705,629
U.S. Pat. No. 5,708,153
U.S. Pat. No. 5,708,154
U.S. Pat. No. 5,714,606
U.S. Pat. No. 5,728,525
U.S. Pat. No. 5,744,305
U.S. Pat. No. 5,763,167
U.S. Pat. No. 5,770,358
U.S. Pat. No. 5,777,092
U.S. Pat. No. 5,789,162
U.S. Pat. No. 5,792,847
U.S. Pat. No. 5,800,992
U.S. Pat. No. 5,807,522
U.S. Pat. No. 5,830,645
U.S. Pat. No. 5,837,196
U.S. Pat. No. 5,847,219
U.S. Pat. No. 5,856,174
U.S. Pat. No. 5,858,988
U.S. Pat. No. 5,859,221
U.S. Pat. No. 5,871,928
U.S. Pat. No. 5,872,232
U.S. Pat. No. 5,876,932
U.S. Pat. No. 5,886,165
U.S. Pat. No. 5,919,626
U.S. Pat. No. 5,922,591
U.S. Pat. No. 6,004,755
U.S. Pat. No. 6,040,193
U.S. Pat. No. 6,087,102
U.S. Pat. No. 6,251,666
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Pat. No. 6,723,509
U.S. patent Ser. No. 09/545,207
U.S. patent Ser. No. 10/667,126
U.S. patent Ser. No. 11/141,707
U.S. Prov. Appln. 60/575,743
U.S. Prov. Appln. 60/649,584
U.S. Prov. Appln. Methods and Compositions Involving miRNA and miRNA Inhibitor Molecules,
EP 266,032
EP 373 203
EP 785 280
EP 799 897
PCT Appln. WO 0168255
PCT Appln. WO 03020898
PCT Appln. WO 03022421
PCT Appln. WO 03023058
PCT Appln. WO 03029485
PCT Appln. WO 03040410
PCT Appln. WO 03053586
PCT Appln. WO 03066906
PCT Appln. WO 03067217
PCT Appln. WO 03076928
PCT Appln. WO 03087297
PCT Appln. WO 03091426
PCT Appln. WO 03093810
PCT Appln. WO 03100448A1
PCT Appln. WO 04020085
PCT Appln. WO 04027093
PCT Appln. WO 09923256
PCT Appln. WO 09936760
PCT Appln. WO 93/17126
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 9743450
PCT Appln. WO 99/35505
PCT Appln. WO0138580
PCT Appln. WO03100012
UK Patent 1,529,202
UK Patent 8 803 000
Ambros, Cell, 107(7):823-826, 2001.
Brennecke et al., Cell, 113(1):25-36, 2003.
Byrd et al., Blood, 100:4325-4336, 2002.
Calin et al., Proc. Natl. Acad. Sci. USA, 99(24):15524-15529, 2002.
Carrington and Ambros, Science, 301(5631):336-338, 2003.
Cummins et al., In: IRT. Nucleosides and nucleosides, La Jolla Calif., 72, 1996.
Denli et al., Trends Biochem. Sci., 28:196, 2003.
Didenko, Biotechniques, 31(5):1106-1116, 1118, 1120-1121, 2001.
Emptage et al., Neuron, 29(1):197-208, 2001.
Esquela-Kerscher and Slack, Nat Rev Cancer, 6(4):259-269, 2006.
Fodor et al., Science, 251:767-777, 1991.
Froehler et al., Nucleic Acids Res., 14:5399-5407, 1986.
Goldstone et al., Blood, 98(5):1302-11, 2001.
Griffey et al., J Mass Spectrom, 32(3):305-13, 1997.
Grimwade, Best. Pract. Res. Clin. Haematol., 14:497-529, 2001.
He et al., Nature, 435(7043):828-833, 2005.
He et al., Proc. Natl. Acad. Sci. USA, 102(52):19075-19080, 2005.
Hiddemann et al., J. Clin. Oncol./17(11): 3569-76, 1999.
Itakura and Riggs, Science, 209:1401-1405, 1980.
Killion et al., BMC. Bioinformatics, 4:32, 2003.
Klostermeier and Millar, Biopolymers, 61(3):159-79, 2001-2002.
Kornberg and Baker, In: DNA Replication, 2d Ed., Freeman, San Francisco, 1992.
Lagos-Quintana et al., Science, 294(5543):853-858, 2001.
Lau et al., Science, 294(5543):858-862, 2001.
Lee and Ambros, Science, 294(5543):862-864, 2001.
Livak and Schmittgen, Methods, 25:402-408, 2001.
Lu et al., Nature, 435(7043):834-838, 2005.
Marcucci et al., J. Clin. Oncol., 23:5705-5717, 2005.
Monti et al., Virchows Arch., 445(3):236-247, 2004.
Mrozek et al., Blood, 06-001149v1, 2006.
Olsen et al., Dev. Biol., 216:671, 1999.
Sambrook et al., In: DNA microaarays: a molecular cloning manual, Cold Spring Harbor Laboratory Press, Cold Spring Harbor, N.Y., 1989, 2001, 2003.
Schaich et al., Br. J. Haematol., 112:300-307, 2001.
Scheit, In: Synthesis and Biological Function, Wiley-Interscience, New York, 171-172, 1980.
Seggerson et al., Dev. Biol., 243:215, 2002.
Xu et al., Curr. Biol., 13(9):790-795, 2003.

1. A method for assessing a pathological condition in a patient comprising measuring an expression profile of one or more miRNA in a sample from the patient, wherein a difference in the expression profile in the sample from the patient and an expression profile of a normal sample is indicative of acute myeloid leukemia (AML).

2. The method of claim 1, where the miRNA is one or more of hsa-miR-29, hsa-miR-126, hsa-miR-21, hsa-miR-99, hsa-miR-125, hsa-miR-16, hsa-miR-20, hsa-miR-10, hsa-miR-199a-AS, hsa-miR-23, hsa-let-7, hsa-miR-106, hsa-miR-181, hsa-miR-15, hsa-miR-223, hsa-miR-425, hsa-miR-449, hsa-miR-513, hsa-miR-494, miR-17-5p, hsa-miR-221, hsa-mir-19, hsa-miR-92, hsa-miR-222, hsa-miR-27a, hsa-miR-24, hsa-miR-26, hsa-miR-191, hsa-miR-30, hsa-miR-103, hsa-miR-107, hsa-miR-146, hsa-miR-30a-5p, hsa-miR-93, hsa-miR-342, hsa-miR-150, hsa-miR-205, hsa-miR-143, or hsa-miR-199a.

3. The method of claim 1, wherein the sample is a blood sample.

4. The method of claim 3, wherein the sample comprises CD34+ cells.

5. The method of claim 1, wherein the difference in expression profile is indicative of a cytogenetic sub-classification of AML.

6. The method of claim 5, wherein the cytogenetic sub-classification of AML is monosomy 7/deletion 7, inv (16), or normal cytogenetic sub-classification.

7. The method of claim 6, wherein reduced expression of one or more miRNA is indicative of a inv(16) cytogenetic sub-classification, wherein the miRNA is one or more of hsa-miR-29, miR-126, miR-21, miR-99, miR-125, miR-16, miR-20, miR-10, miR-199a-AS, miR-23, let-7, miR-106, miR-181, miR-15, or miR26.

8. The method of claim 6, wherein increased expression of one or more miRNA is indicative of an inv (16) cytogenetic sub-classification, wherein the miRNA is miR-223, miR-425, or both miR-223 and miR-425.

9. The method of claim 6, wherein reduced expression of one or more miRNA is indicative of a monosomy 7/deletion 7 cytogenetic sub-classification, wherein the miRNA is one or more of hsa-miR-449, hsa-miR-513, or hsa-miR-494.

10. The method of claim 6, wherein increased expression of one or more miRNA is indicative of a monosomy 7/deletion 7 cytogenetic sub-classification, wherein the miRNA is one or more of hsa-miR-181, miR-126, miR-17-5p, miR-20, miR-23, miR-221, miR-106, mir-19, miR-21, miR-92, miR-16, miR-222, miR-223, miR-27a, miR-24, miR-26, miR-191, miR-30, miR-103, let-7, miR-15, miR-107, miR-146, miR-30a-5p, miR-93, or miR-29.

11. The method of claim 6, wherein reduced expression of one or more miRNA is indicative of a normal cytogenetic sub-classification, wherein the miRNA is one or more of hsa-miR-223, miR-16, miR-23, miR342, miR17-5p, miR-15, miR-150, miR-103, miR-107, miR-106, or miR-20.

12. The method of claim 6, wherein increased expression of one or more miRNA is indicative of a normal cytogenetic sub-classification, wherein the miRNA is one or more of hsa-miR-205, hsa-miR-181, hsa-miR-125, hsa-miR-143, hsa-miR-199a.

13. The method of claim 1, wherein assessing the pathological condition of the patient is diagnosing an AML patient, assessing prognosis of the AML patient, or assessing response of the AML patient to therapy.

14. The method of claim 1, wherein assessing the pathological condition of the patient is assessing prognosis of the AML patient.

15. The method of claim 14, wherein prognosis is defined as an estimated time of survival.

16. The method of claim 14, wherein altered expression of one or more miRNA is prognostic for a patient having AML, wherein the miRNA is one or more of hsa-miR-199a-AS, miR-23, miR-26, miR-148, miR-28, let 71, miR-191, miR-146, miR-422, miR-297, miR-128, miR-93, miR-105, miR-25, miR-150, miR-184, miR-100, miR-199a, miR-148, miR-105, miR-195, miR-341, miR-30a-AS, miR-145, miR-205, miR-126, miR-221, miR-342, miR-335, miR-106, miR-16, miR-22, miR-139, miR-223, or miR-10a

17. The method of claim 14, wherein altered expression of one or more miRNA is prognostic for a patient having monosomy 7/deletion 7 cytogenetic sub-classification, wherein the miRNA is one or more of miR-199a-AS, miR-26, miR-148, miR-28, let 71, miR-191, miR-146, miR-422, miR-297, miR-128, miR-93, miR-105, or miR-25.

18. The method of claim 14, wherein altered expression of one or more miRNA is prognostic for a patient having normal cytogenetic sub-classification, wherein the miRNA is one or more of miR-23, miR-150, miR-184, miR-100, miR-199a, miR-148, miR-105, miR-195, miR-341, miR-30a-AS, miR-199a-AS, miR-145, miR-205, miR-126, miR-221, miR-342, miR-335, miR-106, miR-16, miR-22, miR-139, miR-26, miR-223, or miR-10a.

19-24. (canceled)

25. The method of claim 1, wherein expression of the miRNA is determined by an amplification assay or a hybridization assay.

26-28. (canceled)

29. A kit for assessment of an AML sample by miRNA profiling of a sample comprising, in suitable container means, two or more miRNA hybridization or amplification reagents comprising one or more of hsa-miR-29, hsa-miR-126, hsa-miR-21, hsa-miR-99, hsa-miR-125, hsa-miR-16, hsa-miR-20, hsa-miR-10, hsa-miR-199a-AS, hsa-miR-23, hsa-let-7, hsa-miR-106, hsa-miR-181, hsa-miR-15, hsa-miR-223, hsa-miR-425, hsa-miR-449, hsa-miR-513, hsa-miR-494, miR-17-5p, hsa-miR-221, hsa-mir-19, hsa-miR-92, hsa-miR-222, hsa-miR-27a, hsa-miR-24, hsa-miR-26, hsa-miR-191, hsa-miR-30, hsa-miR-103, hsa-miR-107, hsa-miR-146, hsa-miR-30a-5p, hsa-miR-93, hsa-miR-342, hsa-miR-150, hsa-miR-205, hsa-miR-143, or hsa-miR-199a.

30-32. (canceled)
